(19) United States
(12) Patent Application Publication
(10) Pub. No.: US 2013/0143219 A1
(43) Pub. Date: Jun. 6, 2013

(54) METHODS AND COMPOSITIONS FOR HIGH YIELD, SPECIFIC AMPLIFICATION

(71) Applicants: Aoy Tomita Mitchell, Elm Grove, WI (US); Michael Mitchell, Elm Grove, WI (US); Mats Hidestrand, Brookfield, WI (US)

(72) Inventors: Aoy Tomita Mitchell, Elm Grove, WI (US); Michael Mitchell, Elm Grove, WI (US); Mats Hidestrand, Brookfield, WI (US)

(73) Assignee: Medical College of Wisconsin Inc., Milwaukee, WI (US)

(21) Appl. No.: 13/575,874

(22) PCT Filed: Jan. 28, 2011
(86) PCT No.: PCT/US11/23067

§ 371 Date: Feb. 20, 2013

Related U.S. Application Data
(60) Provisional application No. 61/299,253, filed on Jan. 28, 2010.

(51) Int. Cl. C12Q 1/68; C07H 21/04

(52) U.S. Cl. CPC C12Q 1/6858; C07H 21/04
USPC 435/6.12; 435/91.2; 536/24.33

(57) ABSTRACT

The present invention is directed to methods and compositions for amplifying nucleic acids. Included in the present invention are methods and compositions that amplify nucleic acids with high yield with the formation of unstable target extension products, preferably with minimal or no introduction of allelic bias. Also included in the present invention are high yield, instability primers for use in amplification methods, as multiplexed amplification methods.

### RELATED APPLICATIONS

This application claims the benefit under 35 U.S.C. §119 of U.S. provisional application 61/299253, filed Jan. 28, 2010, the entire contents of which are incorporated herein by reference.

### FIELD OF THE INVENTION

This invention relates, at least in part, to compositions and methods for high yield, specific amplification (HYSA).

### BACKGROUND OF THE INVENTION

Many diagnostic tests, particularly those directed to the detection of minor allele species in a heterogeneous mixture, require amplification of specific genetic material from a sample. For example, detection of single nucleotide polymorphisms from a sample containing a mixture of DNA (for example, a plasma sample from a pregnant female, which contains both maternal and fetal DNA) can require amplification of specific genes or regions of chromosomes in order to conduct the diagnostic tests. However, global amplification methods (such as whole genome amplification) often result in the introduction of non-specific amplification artifacts, incomplete coverage of loci, and the propensity to generate products that are preferentially amplified, resulting in biased representation of genomic sequences in the products of the amplification reaction. Such artifacts and allelic bias can severely compromise tests that rely on characteristics such as copy number or concentration. A sensitive efficient method of amplification that introduces minimal or no allelic bias would thus be of use for any application (such as diagnostic tests or genomic sequencing) that relies on amplification to generate a sufficient quantity of material.

### SUMMARY OF THE INVENTION

It has been surprisingly discovered that primers that create unstable extension products during amplification lead to high yield target sequence amplification with no non-target products. It has also been surprisingly discovered that such primers can be used for amplification over a broad range of temperatures. As a result, the primers provided herein can be used to amplify multiple target sequences in a single reaction, amplify minority alleles within target sequences with high yield, and/or allow for accurate quantification of one or more target sequences. Accordingly, primers that exhibit the above features and methods of their use are provided herein.

In one aspect, the invention provides high yield, instability primers. In one embodiment, such primers comprise an oligonucleotide flap at one terminus of the primer. In another embodiment, the oligonucleotide flap is an AT-rich flap. In still another embodiment, the oligonucleotide flap is a GC-rich flap. In still another embodiment, the oligonucleotide flap is not a GC-rich flap. In yet another embodiment, the oligonucleotide flap does not consist of the sequence as set forth as SEQ ID NO: 15. In a further embodiment, the oligonucleotide flap does not consist of the sequence set forth as SEQ ID NO: 21, SEQ ID NO: 22, SEQ ID NO: 26, SEQ ID NO: 27, SEQ ID NO: 28, SEQ ID NO: 29, SEQ ID NO: 30, SEQ ID NO: 31 and/or SEQ ID NO: 32. In still another embodiment, the oligonucleotide flap is a mismatched sequence. In yet another embodiment, the oligonucleotide flap is at the 5′ terminus of the primer. In a further embodiment, the oligonucleotide flap is at the 3′ terminus of the primer. In one embodiment, the oligonucleotide flap is less than 54 nucleotides in length. In another embodiment, the oligonucleotide flap is less than 30 nucleotides, 25 nucleotides, 20 nucleotides, 15 nucleotides or 10 nucleotides in length. In still another embodiment, the oligonucleotide flap is between 8 and 30 oligonucleotides, 8 and 25 oligonucleotides, 8 and 20 oligonucleotides, 8 and 15 oligonucleotides or 8 and 12 oligonucleotides. In still other embodiments, the oligonucleotide flap is between 12 and 30 oligonucleotides, 12 and 25 oligonucleotides or 12 and 20 oligonucleotides in length.

In one embodiment, the primer and/or the oligonucleotide flap of the primer is not self-annealing (or exhibits minimal self-annealing). Self-annealing, or primer-dimers, can typically be visualized on a gel as a low molecular weight product. In a further embodiment, the primer and/or oligonucleotide flap is not self-annealing and no primer-dimers can be visualized on a gel. In another embodiment, the primer and/or oligonucleotide flap does not form a hairpin loop. In a further embodiment, the primer and/or oligonucleotide flap does not form a hairpin loop with dG less than or equal to 1.5 kcal/mole as measured by using an oligonucleotide analyzer (e.g., such as OligoAnalyzer 3.1 of Integrated DNA Technologies, Coralville, Iowa, which can be found at idtdna.com).

In another embodiment, the high yield, instability primer comprises one or more mismatches within its 5′ region. In one embodiment, the one or more mismatches are contained within the 5′ half of the primer. In another embodiment, at least 5%, 10%, 15%, 20%, 25%, 30%, 40%, 50%, 60%, 70%, 80%, 90% or 95% of the nucleotide bases of the 5′ half of the primer are mismatched relative to a target sequence (i.e., at least a portion of the sequence of a target polynucleotide, the amplification of which is desired). In a further embodiment, 100% of the nucleotide bases of the 5′ half of the primer are mismatched relative to a target sequence.

In another embodiment, the high yield, instability primers when added to a reaction mixture comprising a target polynucleotide template, under conditions that permit replication and amplification of the target polynucleotide template, permit the production of a target extension product at a yield (or efficiency) of greater than 100% but no non-target product. In one embodiment, the yield is greater than 150%, 200%, 250%, 300%, 350%, 400%, 450%, 500%, 600%, 700%, 800%, 900%, 1000%, 1250%, 1500%, 1750%, 2000%, 2250% or 2500%. In one embodiment, the amplification is linear polymerase chain reaction (PCR) amplification. In another embodiment, the yield is over a certain number of cycles. In another embodiment, the yield is for a particular number of cycles.

In another embodiment, no non-target product is visibly observed by gel analysis. In another embodiment, no non-target product is determined by observing the slope of product formation by real-time PCR analysis, whereby the absence of a product with an unusual slope or a slope differing from a target or control template indicates that no non-target extension product is produced.

In a further embodiment, the high yield, instability primers have an annealing temperature that is at or above its calculated melting temperature (e.g., as in Modified

Breslauer's thermodynamics). In yet another embodiment, the high yield, instability primers do not comprise a flap and have an annealing temperature that is at least 1, 2, 3, 4 or 5 degrees above its calculated melting temperature. In a further embodiment, the high yield, instability primers do not have a flap and have an annealing temperature that is 5, 6, 7, 8, 9 or 10 degrees greater than its calculated melting temperature. In another embodiment, such primers amplify a target polynucleotide in the presence of Taq polymerase.

In one embodiment, the high yield, instability primers can anneal and amplify a target polynucleotide at more than one temperature. In another embodiment, the high yield, instability primers can anneal and amplify a target polynucleotide at 2, 3, 4, 5, 6, 7, 8, 9 or 10 or more temperatures. In a further embodiment, the temperatures are within the range of 50-65° C. In another embodiment, the high yield, instability primers can anneal and amplify a target polynucleotide at 50° C. and 55° C.; at 55° C. and 60° C.; at 50° C., 55° C. and 60° C.; or at 55° C., 60° C., and 65° C.

In another embodiment, the high yield, instability primers can anneal and amplify a target polynucleotide with a yield of greater than 100%, 150%, 200%, 250%, 300%, 350%, 400%, 450%, 500%, 600%, 700%, 800%, 900%, 1000%, 1250%, 1500%, 1750%, 2000%, 2250% or 2500%

In another embodiment, the high yield, instability primers anneal to a target polynucleotide at a temperature equal to or greater than 65° C. In still another embodiment, such primers amplify a target polynucleotide in the presence of Phusion® polymerase.

In yet a further embodiment, the high yield, instability primers comprise a sequence selected from the sequences set forth in SEQ ID NOs: 1-32 (or the sequence of the oligonucleotide flaps present therein).

In one aspect, the invention provides methods for amplifying multiple target polynucleotides, which may comprise: (a) adding two or more primers to a reaction mixture that comprises two or more target polynucleotides; and (b) incubating the reaction mixture under conditions that promote replication of the target polynucleotides, thereby amplifying the target polynucleotides, wherein at least one of the primers is a high yield, instability primer. In one embodiment, all of the primers are high yield, instability primers. In another embodiment, some of the primers are high yield, instability primers, while other of the primers are not. In one embodiment, at least one of the high yield, instability primers does not comprise a GC-clamp with the sequence set forth as SEQ ID NO: 15. In another embodiment, all of the high yield, instability primers do not comprise a GC-clamp with the sequence set forth as SEQ ID NO: 15. In one embodiment, at least one of the high yield, instability primers does not comprise a GC-clamp. In another embodiment, all of the high yield, instability primers do not comprise a GC-clamp. In still another embodiment, at least one of the high yield, instability primers do not comprise a flap that consists of the sequence as set forth as SEQ ID NO: 21, SEQ ID NO: 22, SEQ ID NO: 26, SEQ ID NO: 27, SEQ ID NO: 28, SEQ ID NO: 29, SEQ ID NO: 30, SEQ ID NO: 31 or SEQ ID NO: 32. In yet another embodiment, all of the high yield, instability primers do not comprise a flap that consists of the sequence as set forth as SEQ ID NO: 21, SEQ ID NO: 22, SEQ ID NO: 26, SEQ ID NO: 27, SEQ ID NO: 28, SEQ ID NO: 29, SEQ ID NO: 30, SEQ ID NO: 31 or SEQ ID NO: 32. In one embodiment, 2, 3, 4, 5, 6, 7, 8, 9, 10, 12, 15, 17, 20, 25, 30, 35, 40 or more high yield, instability primers are added to a reaction mixture that comprises 3, 4, 5, 6, 7, 8, 9, 10, 12, 15, 17, 20, 25, 30, 35, 40 or more target polynucleotides, respectively. In another embodiment, of the primers that are added to the reaction mixture at least 2%, 5%, 10%, 15%, 20%, 25%, 30%, 40%, 50%, 60%, 70%, 80% or 90% are high yield, instability primers, while the rest of the primers are not. In one embodiment, at least one of the high yield, instability primers can anneal and amplify a target polynucleotide at more than one temperature. In another embodiment, at least 2%, 5%, 10%, 15%, 20%, 25%, 30%, 40%, 50%, 60%, 70%, 80% or 90% of the high yield, instability primers can anneal and amplify a target polynucleotide at 2, 3, 4, 5, 6, 7, 8, 9 or 10 or more temperatures. In a further embodiment, the temperatures are within the range of 50-65° C. In another embodiment, at least 2%, 5%, 10%, 15%, 20%, 25%, 30%, 40%, 50%, 60%, 70%, 80% or 90% of the high yield, instability primers can anneal and amplify a target polynucleotide at 50° C. and 55° C.; at 55° C. and 60° C.; at 50° C., 55° C. and 60° C.; or at 55° C., 60° C., and 65° C.

In another aspect, the invention provides methods of amplifying at least a portion of a target chromosome, which may comprise (a) bringing into contact a set of primers, DNA polymerase, and a target polynucleotide, wherein the target polynucleotide comprises the target chromosome, and wherein at least one of the set of primers is a high yield, instability primer; (b) incubating the target polynucleotide under conditions that promote replication of nucleic acids for a period of time sufficient to amplify the target chromosome.

In another aspect, the invention provides methods for amplifying a minority sequence, which may comprise: (a) adding a high yield, instability primer to a reaction mixture that comprises a target polynucleotide comprising the minority sequence; and (b) incubating the reaction mixture under conditions that promote replication of the target polynucleotide, thereby amplifying the minority sequence. In one embodiment, the high yield, instability primer does not comprise a GC-clamp with the sequence set forth as SEQ ID NO: 15. In another embodiment, the high yield, instability primer does not comprise a GC-clamp. In another embodiment, all of the high yield, instability primers does not comprise a GC-clamp. In still another embodiment, the high yield, instability primer does not comprise a flap that consists of the sequence as set forth as SEQ ID NO: 21, SEQ ID NO: 22, SEQ ID NO: 26, SEQ ID NO: 27, SEQ ID NO: 28, SEQ ID NO: 29, SEQ ID NO: 30, SEQ ID NO: 31 or SEQ ID NO: 32.

In yet another aspect, the invention provides methods for generating unstable target extension products, which may comprise: (a) adding a high yield, instability primer to a reaction mixture that comprises a target polynucleotide template; and (b) incubating the reaction mixture under conditions that promote replication and amplification of the target polynucleotide template, thereby generating the unstable target extension products, wherein the high yield, instability primer is any of the primers provided herein. In one embodiment, the primer is i) a minimal or non-self annealing primer comprising an oligonucleotide flap, ii) has an annealing temperature that is at or above its calculated melting temperature, iii) comprises one or more mismatches within its 5′ region, or iv) can anneal and amplify a target polynucleotide at more than one temperature.

In still another aspect, the invention provide methods for generating unstable target extension products, which may comprise: (a) adding a high yield, instability primer to a reaction mixture that comprises a target polynucleotide template; and (b) incubating the reaction mixture under conditions that promote replication and amplification of the target polynucleotide template, thereby generating the unstable target extension products, wherein the high yield, instability primer does not comprise a flap, and wherein the conditions include an annealing temperature that is greater than the calculated melting temperature of the primer. In another embodiment, the annealing temperature is at least 1, 2, 3, 4, 5, 6, 7, 8, 9 or 10 degrees greater than the calculated melting temperature. In a further embodiment, the annealing temperature is at least 5 degrees greater than the calculated melting temperature. In still a further embodiment, Taq polymerase is added to the reaction mixture for replication and amplification.

In still further aspect, the invention provide methods for generating unstable target extension products, which may comprise: (a) adding a high yield, instability primer to a reaction mixture that comprises a target polynucleotide template; and (b) incubating the reaction mixture under conditions that promote replication and amplification of the target polynucleotide template, thereby generating the unstable target extension products, wherein the high yield, instability primer comprises a flap, and wherein the conditions include an annealing temperature that is less than the calculated melting temperature of the primer (without the flap). In another embodiment, the annealing temperature is at least 1, 2, 3, 4, 5, 6, 7, 8, 9 or 10 degrees less than the calculated melting temperature. In a further embodiment, the annealing temperature is at least 5 degrees less than the calculated melting temperature.

In another embodiment, any of the methods can further comprise determining a relative or absolute amount of the target polynucleotide, such as a minority sequence, in the reaction mixture. In a further embodiment, the relative or absolute amounts are copy number or a concentration.

In still another aspect, the invention provides methods of testing a primer, which may comprise: (a) adding to a terminus of the primer an oligonucleotide flap; (b) testing the primer to determine if it anneals and amplifies a target polynucleotide template at at least two different temperatures; and (c) testing the primer to determine if a non-target polynucleotide extension product is produced at each of the temperatures. In one embodiment, the temperatures are within the range of 50-65° C. In another embodiment, the temperatures are 50° C. and 55° C.; at 55° C. and 60° C.; at 50° C., 55° C. and 60° C.; or at 55° C., 60° C., and 65° C. In still another aspect, the invention provides methods of testing a primer, which may comprise: (a) adding to a terminus of the primer an oligonucleotide flap; (b) testing the primer to determine the yield at which it anneals and amplifies at least one target polynucleotide template; and (c) testing the primer to determine if a non-target polynucleotide extension product is produced. In still another embodiment, the oligonucleotide flap is any of the oligonucleotide flaps provided herein.

In a further aspect, the invention provides methods of testing a primer, which may comprise (a) creating a primer that has an annealing temperature that is at or above its calculated melting temperature; (b) testing the primer to determine if it anneals and amplifies a target polynucleotide template in the presence of Taq polymerase; and (c) testing the primer to determine if a non-target polynucleotide extension product is produced. In one embodiment, the annealing temperature is at least 1, 2, 3, 4 or 5 degrees above its calculated melting temperature. In a further embodiment, the annealing temperature is 5, 6, 7, 8, 9 or 10 degrees greater than its calculated melting temperature. In a further aspect, the invention provides methods of testing a primer, which may comprise (a) creating a primer that has an annealing temperature that is at or above its calculated melting temperature; (b) testing the primer to determine the yield at which it anneals and amplifies at least one target polynucleotide template in the presence of Taq polymerase; and (c) testing the primer to determine if a non-target polynucleotide extension product is produced. In one embodiment, the annealing temperature is at least 1, 2, 3, 4 or 5 degrees above its calculated melting temperature. In a further embodiment, the annealing temperature is 5, 6, 7, 8, 9 or 10 degrees greater than its calculated melting temperature. In still another aspect, the invention provides methods of testing a primer, which may comprise: (a) creating a primer that has an annealing temperature that is at or above its calculated melting temperature; (b) testing the primer to determine if it anneals and amplifies a target polynucleotide template at at least two different temperatures; and (c) testing the primer to determine if a non-target polynucleotide extension product is produced at each of the temperatures. In one embodiment, the temperatures are within the range of 50-65° C. In another embodiment, the temperatures are 50° C. and 55° C.; at 55° C. and 60° C.; at 50° C., 55° C. and 60° C.; or at 55° C., 60° C., and 65° C. In one embodiment, the annealing temperature is at least 1, 2, 3, 4 or 5 degrees above its calculated melting temperature. In a further embodiment, the annealing temperature is 5, 6, 7, 8, 9 or 10 degrees greater than its calculated melting temperature.

In still another aspect, the invention provides methods of testing a primer, which may comprise: (a) creating a primer that comprises one or more mismatches in its 5′ region; (b) testing the primer to determine if it anneals and amplifies a target polynucleotide template at at least two different temperatures; and (c) testing the primer to determine if a non-target polynucleotide extension product is produced at each temperature. In one embodiment, the temperatures are within the range of 50-65° C. In another embodiment, the temperatures are 50° C. and 55° C.; at 55° C. and 60° C.; at 50° C., 55° C. and 60° C.; or at 55° C., 60° C., and 65° C. In still another aspect, the invention provides methods of testing a primer, which may comprise: (a) creating a primer that comprises one or more mismatches in its 5′ region; (b) testing the primer to determine the yield at which it anneals and amplifies at least one target polynucleotide template; and (c) testing the primer to determine if a non-target polynucleotide extension product is produced. In one embodiment, the one or more mismatches are contained within the 5′ half of the primer. In another embodiment, at least 5%, 10%, 15%, 20%, 25%, 30%, 40%, 50%, 60%, 70%, 80%, 90% or 95% of the nucleotide bases of the 5′ half of the primer are mismatched relative to a target sequence.

In one embodiment of any of the methods provided, each of the annealing temperatures is within the range of 50-65° C. In another embodiment, each of the annealing temperatures can be 50° C., 55° C., 60° C. or 65° C.

In any one of the methods or compositions provided herein, the conditions required for amplification may be conditions required for amplification by polymerase chain reaction (PCR). In one embodiment, the PCR is linear PCR.

In another embodiment of any of the methods or compositions provided herein, the yield of the amplification with at least one of the high yield, instability primers is greater than 100%, 150%, 200%, 250%, 300%, 350%, 400%, 450%, 500%, 600%, 700%, 800%, 900%, 1000%, 1250%, 1500%, 1750%, 2000%, 2250% or 2500%. In still another embodiment, in addition, no non-target product is produced. In one embodiment, the amplification is linear amplification. In another embodiment, the yield is for a particular cycle or over a certain number of cycles. In still a further embodiment, the steps for determining the yield can comprise the steps of any of the methods provided herein. In one embodiment the steps comprise those depicted in FIG. 1.

In one embodiment of any of the methods or compositions provided herein, the amplification results in minimal allelic bias.

In another embodiment of any of the methods or compositions provided herein, the relative or absolute amount of at least one of the target polynucleotides may be determined. The relative amount may be relative to another of the target polynucleotides. In still another embodiment, the amount is a copy number or concentration.

In one embodiment of any of the methods or compositions provided herein, the target polynucleotide(s) comprises a whole genome.

In another embodiment of any of the methods or compositions provided herein, the target polynucleotide(s) may comprise a minority sequence. The minority sequence may be a fetal allele, a transplant donor-specific sequence, or a microorganism-specific sequence. The minority sequence may also be one that comprises one or more somatic mutations. The somatic mutations may be associated with a disease, such as cancer. “Minority sequence” may also refer to a nucleic acid that is of a non-ideal quality and/or quantity in sample.

In a further embodiment of any of the methods or compositions provided herein, the target polynucleotide(s) may be from plasma or blood. The plasma or blood sample may be from a pregnant female. The plasma or blood sample may comprise fetal and maternal DNA, wherein the target polynucleotide(s) comprises fetal DNA.

In yet a further embodiment of any of the methods or compositions provided herein, the target polynucleotide(s) may comprise or be located on a chromosome. The chromosome may be chromosome 19 or 21.

In one embodiment of any of the methods or compositions provided herein, a polymerase may be added to a reaction mixture. In another embodiment, the polymerase is Taq polymerase, Pfu polymerase or ^{Phusion}® polymerase except where the polymerase is specifically noted.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 is a schematic illustration of an experimental set-up for demonstrating the efficiency, or yield, of HELP amplification. FIG. 1A shows the reaction conditions for the control experiments and FIG. 1B shows the reaction conditions for the HELP amplification.

FIG. 2 is a bar graph demonstrating the fold-change or yield following linear amplification, where either no 5′-flap or three different 5′-flaps were used and quantified via quantitative real-time PCR.

FIG. 3 is a thermodynamic melting profile of the different 5′-flaps listed in FIG. 2.

FIG. 4 is a schematic illustration of an embodiment of the invention.

FIG. 5 is a schematic illustration of multiple primers with 5′-flaps bound to and amplifying various targets on the same chromosome in a multiplexed reaction. The top panel depicts a subchromosomal view and the bottom panel depicts a chromosomal view.

FIG. 6 is an electropherogram showing allelic bias that results using ligation-mediated PCR (FIG. 6A) and the lack of allelic bias from HELP amplification (FIG. 6B).

FIG. 7 is a photograph of an electrophoresis gel showing target-specific amplification products using LPCR reactions with Taq polymerase and the renin gene as starting template.

FIG. 8 is a photograph of an electrophoresis del showing target-specific amplification products using LPCR reactions with Taq polymerase and the renin gene as starting template.

FIG. 9 shows the yield of the LPCR reactions as measured using target-specific real-time PCR.

FIG. 10 is a graph of an amplification curve, showing that from LPCR using primers without an oligonucleotide flap creates non-target product at low temperatures. High molecular weight non-target amplification is shown.

FIG. 11 is a graph of an amplification curve, showing that LPCR using primers without an oligonucleotide flap creates non-target product at low temperatures. High molecular weight non-target amplification is shown.

FIG. 12 is a graph of an amplification curve, showing results of LPCR using primers with a 12-mer AT+C tail (FIG. 12A) and quantification of those results (FIG. 12B).

FIG. 13 is a graph of an amplification curve, showing results of LPCR using primers with a 26-mer AT1 tail (FIG. 13A) and quantification of those results (FIG. 13B).

FIG. 14 is a graph of an amplification curve, showing results of LPCR using primers with a 26-mer AT+C tail (FIG. 14A) and quantification of those results (FIG. 14B).

FIG. 15 is a graph of an amplification curve, showing results of LPCR using primers with a 28-mer AT tail (FIG. 15A) and quantification of those results (FIG. 15B).

FIG. 16 is a graph of an amplification curve, showing results of LPCR using primers with a 12-mer GC tail (FIG. 16A) and quantification of those results (FIG. 16B).

FIG. 17 is a graph of an amplification curve, showing results of LPCR using primers with a 12-mer AGCT tail (FIG. 17A) and quantification of those results (FIG. 17B). This particular primer creates non-target product at 50° C., and correct product at both 55° C. and 60° C.

FIG. 18 is a graph of an amplification curve, showing results of LPCR using primers with a 54-mer GC tail (FIG. 18A) and quantification of those results (FIG. 18B).

FIG. 19 is a graph of an amplification curve, showing results of LPCR using primers with a 54-mer AT tail (FIG. 19A) and quantification of those results (FIG. 19B).

FIG. 20 is a graph of an amplification curve, showing results of LPCR using primers with a 54-mer AT tail with extra Cs (FIG. 20A) and quantification of those results (FIG. 20B).

FIG. 21 is a graph of an amplification curve, showing results of LPCR with Taq Tandem SNP region ATM102-103 (SYBR green detection with prATM626/627).

FIG. 22 is a graph of the quantification of the results in FIG. 21.

FIG. 23 is a photograph of an electrophoresis gel showing amplification products from LPCR reactions with Pfu polymerase (FIG. 23A) and a graph of an amplification curve (FIG. 23B).

FIG. 24 is a photograph of an electrophoresis gel showing a comparison of LPCR using Pfu polymerase or Phusion polymerase.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention. The citation of any reference herein is not an admission that the reference is indeed prior art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details.

In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

The one-letter codes “A”, “C”, “T” and “G” refer to adenosine, cytosine, thymine and guanine, respectively.

“Allelic bias” refers to a non-uniform amplification of a mixture of nucleic acids, such that certain alleles are preferably amplified over others. Allelic bias is often seen in whole genome amplification techniques and can introduce errors into diagnostic tests utilizing the amplification products.

“Conditions that promote replication” refers to standard or modified amplification conditions, including temperature (e.g., DNA melting or denaturation temperature, primer annealing temperature, primer extension/elongation temperature), reaction mixture volume, and timing and number of amplification cycles. In one aspect, the conditions are those required for PCR or linear PCR.

“High yield, instability primer” refers to a primer that gives a minimal level of amplification yield and that does not produce non-target products. “Efficiency” and “yield” may be used interchangeably herein and refer to the difference between the target copy number before and after amplification divided by the cycle number or number of cycles. When referring to a primer that permits high yield amplification, it is to be understood that the minimum level of amplification yield is 100%. In embodiments, however, the yield is greater than 100%, 150%, 200%, 250%, 300%, 350%, 400%, 450%, 500%, 600%, 700%, 800%, 900%, 1000%, 1250%, 1500%, 1750%, 2000%, 2250% or 2500%. In one embodiment, the yield is calculated by the cycle number. In another embodiment, the yield is calculated by the number of cycles.

A nucleic acid is “homologous” to another if there is some degree of sequence identity between the two. Preferably, a homologous sequence will have at least about 85% sequence identity to the reference sequence, preferably with at least about 90% to 100% sequence identity, more preferably with at least about 91% sequence identity, with at least about 92% sequence identity, with at least about 93% sequence identity, with at least about 94% sequence identity, more preferably still with at least about 95% to 99% sequence identity, preferably with at least about 96% sequence identity, with at least about 97% sequence identity, with at least about 98% sequence identity, still more preferably with at least about 99% sequence identity, and about 100% sequence identity to the reference amino acid or nucleotide sequence.

An “isolated” molecule, such as an isolated nucleic acid, is one which has been identified and separated and/or recovered from a component of its natural environment. The identification, separation and/or recovery are accomplished through techniques known in the art, or readily available modifications thereof.

“Non-target products” refers to amplification of an undesired sequence, such as the formation of primer dimers. In one embodiment, the non-target product is incorrect, high molecular weight amplification products, which interfere with the amplification process.

The terms “nucleic acid” and “nucleotide” and “polynucleotide” and “oligonucleotide” are used interchangeably and refer to DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole; such nucleic acids may also be referred to as bases of non-naturally occurring nucleotide mono- and higher- phosphates. Modifications can also include 3′ and 5′ modifications such as capping with a quencher, a fluorophore or another moiety.

“Reaction mixture” refers to a composition that comprises the target polynucleotide(s) (or target template) for amplification. Reaction mixtures can also comprise primer(s), polymerase, deoxynucleotide triphosphates (dNTPs) and salt.

The present invention is directed to methods and compositions for amplifying nucleic acids with high yield such that unstable extension products are produced. It has been surprisingly discovered that primers that create unstable extension products during amplification lead to high yield target sequence amplification with no non-target products. It has also been surprisingly discovered that such primers can be used for amplification over a broad range of temperatures. As a result, the primers provided herein can be used for amplification of multiple target sequences in a single reaction, amplify minority target sequences with high yield, and/or allow for accurate quantification of one or more target sequences. Accordingly, primers that exhibit the above features and methods of their use are provided herein.

An embodiment of an amplification method of the present invention is referred to herein as High Yield, Specific Amplification. As will be appreciated, although the present invention is primarily described herein in terms of PCR, other amplification methods known in the art are encompassed by and can be used in accordance with the present invention. As will be further appreciated, although the discussion herein focuses primarily on methods and compositions involving DNA, it would be within the skill of one in the art to alter reaction conditions and reagents discussed herein for amplification of any nucleic acid.

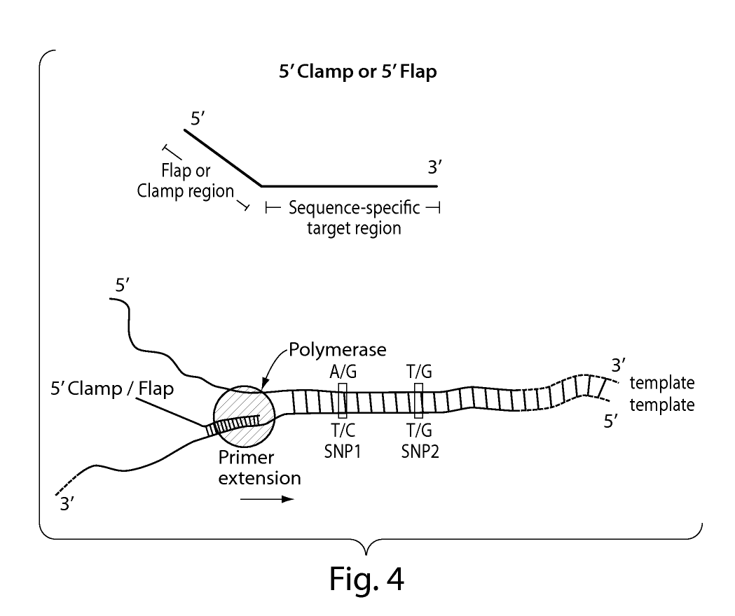

In one aspect, the present invention provides methods and compositions for nucleic acid amplification method using high yield, instability primers. In one aspect, primers may comprise an oligonucleotide flap. Oligonucleotide flaps of the invention comprise a string or sequence of nucleotides that are generally located on one terminus of a primer. Oligonucleotide flaps lack complementarity to the target sequence that is being amplified (see FIG. 4), such that they remain free from the target sequence to which the remainder of the primer is annealed. The oligonucleotide flaps of the invention improve the efficiency of amplification methods by reducing primer-primer interactions and by reducing allelic bias during amplification. Oligonucleotide flaps may also be referred to in the art as clamps and/or tails.

In one embodiment, the flap is a five prime (5′) flap. In a further embodiment, 5′ flaps of the present invention comprise structural features that improve primer extension efficiency. In a still further embodiment, oligonucleotide flaps of primers of the present invention are AT-rich. In further embodiments, oligonucleotide flaps of the invention are GC-rich. In still a further embodiment, the oligonucleotide flaps of the invention are a mismatched sequence.

Oligonucleotide flaps of the present invention may comprise fewer than 54 nucleotides, fewer than 30 nucleotides, fewer than 25 nucleotides, fewer than 20 nucleotides, fewer than 15 nucleotides, or fewer than 10 nucleotides. In some embodiments, oligonucleotide flaps may comprise about 10 to about 20 nucleotides, about 20 to about 30 nucleotides, about 30 to about 40 nucleotides, or about 40 to about 50 nucleotides. In one embodiment, oligonucleotide flaps consist of 12 nucleotides. In another embodiment, oligonucleotide flaps consist of 18 nucleotides. In yet another embodiment, oligonucleotide flaps consist of 26 nucleotides. In still another embodiment, oligonucleotide flaps of the present invention are not “GC-clamps” consisting of greater than or equal to 54 nucleotides. In still another embodiment, oligonucleotide flaps do not consist the sequence as set forth as SEQ ID NO: 15. As will be appreciated, an “X-mer” oligonucleotide is an oligonucleotide that has X number of nucleotides—for example, a 10-mer oligonucleotide has 10 nucleotides.

In yet further embodiments, oligonucleotide flaps of the invention are 12-mer or 26-mer oligonucleotides attached to primers of varying lengths from about 5 to about 150 nucleotides long. In some embodiments, the primers without the oligonucleotide flaps are about 5 to about 10, about 10 to about 15, about 15 to about 20, about 20 to about 25, or about 25 to about 30, about 30 to about 35, about 35 to about 40, about 40 to about 45, or about 45 to about 50 nucleotides in length.

In some embodiments of the invention, high yield, instability primers comprise mismatch sequences in their 5′ region. In some embodiments, the mismatch sequences are within the 5′ half of the primer. Mismatch sequences are those that differ from the sequences present in a target polynucleotide. A mismatch may be a nucleotide that is substituted for a different nucleotide. In some embodiments, a mismatch is a A:T, A:C, A:G, T:A, T:C, T:G, C:A, C:T, C:G, G:A, G:C, or G:T conversion. A mismatch in the 5′ half of a primer refers to any nucleotide substitution in the amino terminal (N-terminal) half of the primer. In some embodiments, the primer may comprise 1 mismatch nucleotide. In other embodiments the primer may comprise 2, 3, 4, or 5 mismatch nucleotides. In another embodiment, at least 5%, 10%, 15%, 20%, 25%, 30%, 40%, 50%, 60%, 70%, 80%, 90% or 95% of the nucleotides of the 5′ half of the primer are mismatched relative to a target sequence.

In other embodiments of the invention, high yield, instability primers or flaps exhibit minimal to no self-annealing. Self-annealing refers to the ability of the primer or flaps to fold back and anneal to itself, thereby forming a hairpin loop. Self-annealing also refers to the ability of one primer or flap to anneal to a second primer or flap having a similar nucleic acid sequence. Herein, a primer or flap that exhibits no self-annealing properties refers to a primer or flap that does not anneal to itself or another primer or flap at a detectable frequency or level. A detectable frequency or level may be determined by gel electrophoresis. The absence of a visible band (e.g., as visible by the naked eye) on an electrophoretic gel is indicative of minimal to no self-annealing. Self-annealing, or primer-dimers, can typically be visualized on a gel as a low molecular weight product. In a further embodiment, the primer and/or flap is not self-annealing and no primer-dimers can be visualized on a gel. In another embodiment, the primer and/or flap does not form a hairpin loop. In a further embodiment, the primer and/or flap does not form a hairpin loop with dG less than or equal to 1.5 kcal/mole as measured by using an oligonucleotide analyzer (e.g., such as OligoAnalyzer 3.1 of Integrated DNA Technologies, Coralville, Iowa, which can be found at idtdna.com).

In further embodiments, high yield, instability primers are able to anneal to target polynucleotides over a range of annealing temperatures. In one embodiment, a range of annealing temperatures refers to more than one temperature within 10° C. of the calculated annealing temperature of the primer. In one embodiment, the high yield, instability primers are able to anneal to one or more target polynucleotides at at least two different temperatures. In one embodiment, the temperatures are within the range of 50-65° C. In another embodiment, each of the temperatures is 50° C., 55° C., 60° C. or 65° C.

High yield, instability primers of the present invention are advantageous for use in amplification methods, as use of these primers results in no formation of non-target products, such as high molecular weight molecules or by unusual or different amplification slopes during real-time PCR detection. The absence of non-target products may be assessed by gel electrophoresis. The absence of a visible band on an electrophoretic gel is indicative of no production of non-target products. The absence of non-target products may also be assessed by real-time PCR, as non-target product, such as high molecular weight molecules, often has a different or unusual real-time PCR slope from the slope of a target or control template.

In a further aspect, the amplification is a linear amplification, meaning that the DNA template is amplified in only one direction using a single primer—because there is no partner primer, the amplification does not increase exponentially but instead increases linearly. Use of the high yield, instability primers of the present invention can result in greater than 100% yields of amplification product (i.e., greater than 100% efficiency), suggesting multiple primer initiations per parent nucleic acid sequence. In some embodiments yield of amplification product may be about 100% to about 150%, about 150% to about 200%, about 200% to about 250%, about 250% to about 300%, about 300% to about 350%, about 350% to about 400%, about 400% to about 450%, about 450% to about 500%, about 500% to about 750%, about 750% to about 1000%, about 1000% to about 1250%, about 1250% to about 1500%, about 1500% to about 1750%, about 1750% to about 2000%, about 2000% to about 2500%, about 2500% to about 3000%, or more. In some embodiments, amplification yield may be measured by real-time PCR of template DNA, where no polymerase in a PCR reaction represents the starting number of polynucleotide template copies in an experiment, and the amplified copies are shown by a shift of the curve to the left.

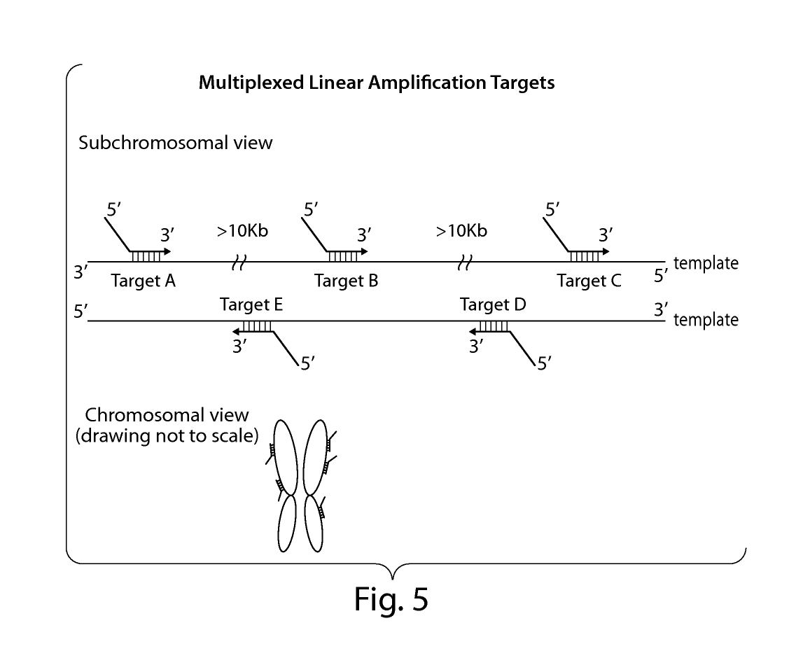

In a still further aspect, the amplification is a Multiplexed Linear Amplification in which multiple targets are amplified in the same reaction, thus further simplifying and streamlining the process of global amplification. In embodiments in which the amplification method is a linear amplification, more high yield, instability primers can be multiplexed together in the same reaction, because there are no partner primers for any given target of interest and these primers can amplify polynucleotides over a range of annealing temperatures. In conventional multiplex reactions, a maximum of about 10 primer pairs can be multiplexed before aberrant primer interactions interfere with PCR targets of interest. In contrast, methods of the present invention allow about 10 to about 40 primers to be multiplexed without triggering aberrant primer interactions. As shown in FIG. 5, multiple primers, for example, primers with 5′-flaps, can bind to and amplify multiply various targets on the same chromosome or other target polynucleotide in a multiplexed reaction. The top panel of FIG. 5 depicts a subchromosomal view and the bottom panel depicts a chromosomal view.

The amplification methods provided herein are of particular use in applications in which avoiding allelic bias in amplification products is of importance, such as whole genome amplification and molecular diagnostics for detection of chromosomal abnormalities. The amplification methods provided herein are also of use in applications in which a starting sample of nucleic acids is of limited volume and/or of poor quality. Nucleic acids having limited volume or poor quality are also referred to herein as minority sequences. Minority sequences include, but are not limited to, fetal alleles, transplant donor-specific sequences (i.e., a sequence that is associated with a donor tissue or organ and not of the transplant recipient), microorganism-specific sequences (i.e., a sequence of a nucleic acid of a microorganism, such as a virus, bacteria, fungus, etc.), and sequences having one or more somatic mutations (i.e., a nucleic acid sequence in which a somatic mutation is present). The somatic mutations may be associated with disease, such as cancer. Minority sequences also include nucleic acids that are of a non-ideal quality and/or quantity in a sample.

The present invention provides methods and compositions for amplification. The following sections describe exemplary embodiments of reagents and methods of use in such amplification. One of skill in the art will understand that the following embodiments can be modified according to standard methods known in the art, and that those modifications are encompassed by the presently described invention.

The methods and compositions of the present invention can result in an amplification of target nucleic acids with minimal allelic bias. By “minimal allelic bias” is meant that the resultant amplification product shows less than about 2% coefficient of variation (CV) allelic bias. Minimal allelic bias also refers to less than a 0.25% difference in amplification efficiency per cycle between alleles. Minimizing and/or eliminating allelic bias is of particular importance in molecular diagnostics methods, because allelic bias can undermine results that rely on determining the copy number of alleles. Methods of the present invention further allow correction of any quantifiable allelic bias that may be produced using primers of the invention. For example, certain primers of the invention may introduce some allelic bias, but that allelic bias would be consistent and predictable when using the methods of the present invention, and would therefore permit correction for any such bias downstream. As such, even if some allelic bias is introduced using methods and compositions of the invention, such bias could be corrected in the final product.

The methods and compositions of the present invention can be used in any nucleic acid amplification method known in the art and described herein. Such amplification methods include polymerase chain reaction (PCR), ligase chain reaction (LCR), ligase detection reaction

(LDR), ligation followed by Q-replicase amplification, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3.sup.rd Edition; Ausbel et al.; PCR Primer: A Laboratory Manual, Diffenbach, Ed., Cold Spring Harbor Press (1995).

The methods and compositions of the present invention can be applied to any sample containing nucleic acids. As will be appreciated, the sample may comprise any number of substances, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; purified samples, such as purified genomic DNA, RNA, proteins, etc.; raw samples (bacteria, virus, genomic DNA, etc.); as will be further appreciated by those in the art, virtually any experimental manipulation may be conducted on the sample prior to application of the present invention. In some embodiments, samples used in accordance with the present invention are obtained from a pregnant female and include both maternal and fetal nucleic acids. Such samples can include without limitation maternal blood, maternal urine, maternal sweat, maternal cells, as well as cell-free nucleic acids.

In one aspect, the present invention provides high yield, instability primers that are of use in the amplification methods provided herein. These primers can provide specific, highly efficient amplification with no non-target product formation, and, preferably, minimal or no allelic bias.

It will be appreciated that, as with any nucleic acid, primers can comprise ribonucleotides, deoxynucleotides, modified ribonucleotides, modified deoxyribonucleotides, modified phosphate-sugar-backbone oligonucleotides, nucleotide analogs, or combinations thereof. For some illustrative teachings of various nucleotide analogs etc, see Fasman, 1989, Practical Handbook of Biochemistry and Molecular Biology, pp. 385-394, CRC Press, Boca Raton, Fla., Loakes, N.A.R. 2001, vol 29:2437-2447, and Pellestor et al., Int J Mol. Med. 2004 April; 13(4):521-5), references cited therein, and recent articles citing these reviews.

In one aspect, primers of the present invention include oligonucleotide flaps. Such flaps improve the efficiency of amplification methods by forming unstable extension products. Oligonucleotide flaps may be attached to the 3′ terminus, the 5′ terminus, or both the 3′ and 5′ terminus of a primer. In specific embodiments, oligonucleotide flaps are 5′ flaps, meaning they are attached to the 5′ terminus of the primer. In further embodiments, oligonucleotide flaps are inserted within a primer or are not part of the linear primer sequence but are instead attached to the primer through modifications of the primer backbone.

In some embodiments, oligonucleotide flaps are attached to primers through a linker. As will be appreciated, such linkers may comprise anything that joins the oligonucleotide flap to the remainder of the primer. In specific embodiments, linkers of use in the primers of the present invention comprise a sequence of nucleotides. In further embodiments, linkers of use in the present invention can include without limitation substituted or unsubstituted alkyl (such as alkane or alkene linkers of from about C20 to about C30), substituted or unsubstituted heteroalkyl, substituted or unsubstituted aryl, substituted or unsubstituted heteroaryl, substituted or unsubstituted cycloalkyl, and substituted or unsubstituted heterocycloalkyl. In still further exemplary embodiments, linkers of the invention may include without limitation poly(ethylene glycol) (PEG) groups, saturated or unsaturated aliphatic structures comprised of single or connected rings, amino acid linkers, peptide linkers, nucleic acid linkers, PNA, LNA, as well as linkers containing phosphate or phosphonate groups. Any combination of the above-described linkers may also be of use in primers of the present invention. In specific embodiments, oligonucleotide flaps of the invention are not attached to the primer through a linker

In a specific embodiment, primers of the present invention include AT-rich flaps. In another embodiment, the flap is not AT-rich. By “AT-rich flap” as used herein is meant a portion of the primer at one terminus (generally the 5′ terminus) that has a sequence comprising at least 50% A′s and/or T′s. In a further embodiment, AT-rich flaps of the present invention comprise about 50-100%, 55-95%, 60%-90%, 65%-85%, and 70%-80% A's and/or T's. In other embodiments, primers of the present invention include GC-rich flaps. By “GC-rich flap” as used herein is meant a portion of the primer at one terminus (generally the 5′ terminus) that has a sequence comprising at least 50% G′s and/or C′s. In a further embodiment, GC-rich flaps of the present invention comprise about 50-100%, 55-95%, 60%-90%, 65%-85%, and 70%-80% G's and/or C's. In other embodiment, the primers do not comprise GC-rich flaps. In one embodiment, the primers do not comprise a GC-flap with the sequence as set forth as SEQ ID NO: 15. In still another embodiment, the primers comprise a mismatched flap. In one embodiment, the flap may comprise 2, 3, 4, or 5 mismatch nucleotides (relative to a target sequence). In another embodiment, at least 5%, 10%, 15%, 20%, 25%, 30%, 40%, 50%, 60%, 70%, 80%, 90% or 95% of the nucleotides of the flap are mismatched relative to a target sequence.

Without being bound by theory, it is believed that oligonucleotide flaps of the invention create a condition of instability of primer binding to the template. This instability facilitates the dissociation or “unzipping” of the newly formed extension product from the template, thereby freeing the template for additional amplifications.

In another aspect, the primers of the present invention are primers with mismatched sequences. In some embodiments of the invention, high yield, instability primers comprise mismatch sequences in their 5′ region. In some embodiments, the mismatch sequences are within the 5′ half of the primer. Mismatch sequences are those that differ from the sequences present in a target polynucleotide. A mismatch may be a nucleotide that is substituted for a different nucleotide. In some embodiments, a mismatch is a A:T, A:C, A:G, T:A, T:C, T:G, C:A, C:T, C:G, G:A, G:C, or G:T conversion. A mismatch in the 5′ half of a primer refers to any nucleotide substitution in the amino terminal (N-terminal) half of the primer. In some embodiments, the primer may comprise 1 mismatch nucleotide. In other embodiments the primer may comprise 2, 3, 4, or 5 mismatch nucleotides. In another embodiment, at least 5%, 10%, 15%, 20%, 25%, 30%, 40%, 50%, 60%, 70%, 80%, 90% or 95% of the nucleotides of the 5′ half of the primer are mismatched relative to a target sequence.

In a further embodiment, primers of the present invention comprise the sequences provided in Tables 1, 2 and 4 or at least the sequences of the flaps contained therein. In a still further embodiment, primers of the present invention have from about 70% to about 100% sequence identity to primers that comprise the sequences provided in Tables 1, 2 and 4 or at least the sequences of the flaps contained therein. In a still further embodiment, primers of the present invention have from about 75% to about 95%, from about 80% to about 90% and from about 85% to about 89% sequence identity to primers that comprise the sequences provided in Tables 1, 2 and 4 or at least the sequences of the flaps contained therein. In a further embodiment, flaps of the present invention have from about 70% to about 100% sequence identity to flaps provided in Tables 1, 2 and 4. In a still further embodiment, flaps of the present invention have from about 75% to about 95%, from about 80% to about 90% and from about 85% to about 89% sequence identity to flaps provided in Tables 1, 2 and 4. It follows that primers comprising these flaps and those that share the above sequence identity are provided.

TABLE 1
Amplification primers (flap sequences are in lowercase letters;
target sequence specific regions of the primers are capitalized)

| Primer | |
|---|---|
| name | Sequence |
| ATM 98 | gccgcctgcagcccgcgccccccgtgcccccgccccgccgccggcccgggcgccCAGTGTTTGGAAATTG |
| | TCTG (SEQ ID NO: 1) |
| ATM 103 | gccgcctgcagcccgcgccccccgtgcccccgccccgccgccggcccgggcgccTCTTCCACCACACCAA |
| | TC (SEQ ID NO: 2) |
| ATM 164 | gccgcctgcagcccgcgccccccgtgcccccgccccgccgccggcccgggcgccAAAAGATGAGACAGGC |
| | AGGT (SEQ ID NO: 3) |
| ATM 168 | gccgcctgcagcccgcgccccccgtgcccccgccccgccgccggcccgggcgccgtgggAGCACTGCAGG |
| | TA (SEQ ID NO: 4) |
| ATM 179 | gccgcctgcagcccgcgccccccgtgcccccgccccgccgccggcccgggcgccAATTATTATTTTGCAG |
| | GCAAT (SEQ ID NO: 5) |
| ATM 91 | gccgcctgcagcccgcgccccccgtgcccccgccccgccgccggcccgggcgccTTCAAATTGTATATAA |
| | GAGAGT (SEQ ID NO: 6) |
| ATM 139 | gccgcctgcagcccgcgccccccgtgcccccgccccgccgccggcccgggcgccATTGTTAGTGCCTCTT |
| | CTGCTT (SEQ ID NO: 7) |
| ATM 205 | gccgcctgcagcccgcgccccccgtgcccccgccccgccgccggcccgggcgcctcagacttgaagtcca |
| | ggat (SEQ ID NO: 8) |
| ATM 189 | gccgcctgcagcccgcgccccccgtgcccccgccccgccgccggcccgggcgccaaggatagagatatac |
| | agatgaatgga (SEQ ID NO: 9) |
| ATM 82 | gccgcctgcagcccgcgccccccgtgcccccgccccgccgccggcccgggcgccATAGCCTGTGAGAATG |
| | CCTA (SEQ ID NO: 10) |
| No Tail | GCCACAGAACCTCAGTGGAT (SEQ ID NO: 11) |
| GC tail | gccgcctgcagcccgcgccccccgtgcccccgccccgccgccggcccgggcgccGCCACAGAACCTCAGT |
| | GGAT (SEQ ID NO: 12) |
| AT 1 tail | AttattCatGatttatattttttaCatttttattttattattaatttaaatattGCCACAGAACCTCAGT |
| | GGAT (SEQ ID NO: 13) |
| AT 2 tail | attatttattttatttttgattatttatttatttttcattatttattttatttttGCCACAGAACCTCAG |
| | TGGAT(SEQ ID NO: 14) |

TABLE 2
Flap sequences

| Primer | |
|---|---|
| name | Sequence |
| GC ATM 291 | gccgcctgcagcccgcgccccccgtgcc |
| | cccgccccgccgccggcccgggcgcc |
| | (SEQ ID NO: 15) |
| AT-1 ATM 375 | attattCatGatttatattttttaCatt |
| | tttattttattattaatttaaatatt |
| | (SEQ ID NO: 16) |
| AT-2 ATM 377 | attatttattttatttttGattatttat |
| | ttatttttCattatttattttattttt |
| | (SEQ ID NO: 17) |
| ½ AT-1 | attattCatGatttatattttttaCa |
| | (SEQ ID NO: 18) |
| ½ AT-1 extra | attattCatGCtttatattttttaCa |
| C | (SEQ ID NO: 19) |
| AT-28′mer | AttatGCatattttatattttttaCatt |
| | (SEQ ID NO: 20) |
| AT-12′mer | aataaatCataa (SEQ ID NO: 21) |
| ATM 457 | |
| AT-9′mer | aaatCataa (SEQ ID NO: 22) |
| GC 12′mer | gccgcctgcacg (SEQ ID NO: 23) |
| GTCA 12′mer | gtcacgtatcga (SEQ ID NO: 24) |
| ATM 463 | |
| AT no C | aataaataataa (SEQ ID NO: 25) |
| 12′mer | |

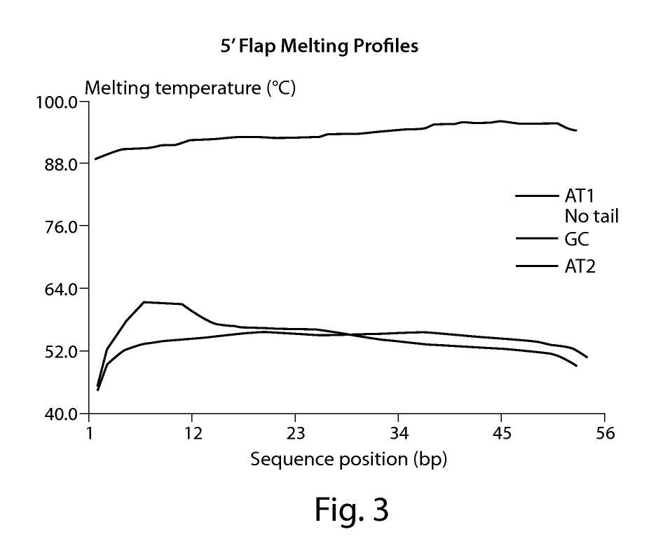

In a still further embodiment, primers of the present invention further comprise structural features that improve the primers' efficiency in amplification reactions. These structural features include specific sequences, including linker sequences. These structural features further include an approximately 5-10° C. difference in melting temperature within the 5′-flap (See FIG. 3).

In still further embodiments, primers of the present invention further include labels. Such labels and methods of attaching such labels to primers are well known in the art. In yet further embodiments, primers of the present invention include one or more “tail” sequences. Such tail sequences are non-specific sequences that may further serve to destabilize primer hybridization and thus improve the efficiency of amplification reactions. In some embodiments, tail sequences are added to primers and are the same sequence. In some embodiments, multiple tail sequences are used to amplify a region of interest.

As will be appreciated, primers of a wide range of lengths are of use in and encompassed by the present invention. In one embodiment, primers of the present invention have a length of from about 30 nucleotides to about 150 nucleotides. In a further embodiment, primers of the present invention have a length of from about 40 to about 140, from about 50 to about 130, from about 60 to about 120, from about 70 to about 110, from about 80 to about 100, and from about 85 to about 95 nucleotides. These lengths may include oligonucleotide flaps or may be in addition to the oligonucleotide flaps of the invention.

Amplification methods of the present invention utilize reagents known in the art to be of use in the amplification of nucleic acids. For example, buffers, salts, and polymerase enzymes generally used in PCR and other nucleic acid amplification methods are also of use in amplification. Reagents that might be added to further improve amplification, particularly primer extension efficiency, include without limitation additives such as bovine serum albumin (BSA), betaine, magnesium and the like.

In one aspect, amplification methods of the present invention are multiplexed, meaning that such amplification methods are conducted on multiple samples at the same time. As mentioned elsewhere herein, amplification methods of the present invention are also multiplexed in that multiple primer pairs are used in the amplification of one or more samples of target nucleic acids. The ability to scale amplification up to amplify multiple samples using multiple primer and/or primer pairs makes amplification particularly amenable to high throughput and automated applications.

In further embodiments, the present invention provides methods for improving the efficiency of primers. In some embodiments, altering concentrations of primers used in multiplex reactions affects their efficiency in priming the amplification reaction. In specific embodiments, the concentrations of the primers comprising are decreased in accordance with the number of different primers utilized in a particular multiplexed amplification reaction. In such embodiments, the more primers that are added, the lower the overall concentration of each primer. For example, two primers used in a standard PCR reaction are generally used at a [1×] concentration. In contrast, in a multiplexed linear reaction utilizing 10 primers, each primer would be used at a [⅕×] concentration.

In some embodiments, multiplexed amplification reactions of the present invention utilize about 2 to about 24 primers in multiplexed linear amplification reactions and about 2 to about 10 primer pairs in non-linear amplification reactions. In further embodiments, about 40 primers are used for multiplexed linear amplification and about 15 to about 20 primers pairs are used in exponential amplification reactions.

Amplification methods and compositions of the present invention can be used in any situation where amplification with minimal allelic bias is of importance. For example, sequencing reactions and molecular diagnostic tests, which often use and/or rely on information related to copy number or concentration of certain genes or nucleic acid sequences present in a sample, can produce erroneous results if allelic bias is present in the sample. Amplification methods are also of use for applications where the starting sample of nucleic acid is of a non-ideal quality and/or quantity. Amplification methods are also of use in applications requiring rare variants analysis and accurate allelic frequency measurements.

In a further aspect, amplification methods of the present invention are used in conjunction with other methods and assays known in the art, including nucleic acid sequencing applications and molecular diagnostic tests. Amplification can be conducted prior to or simultaneously with such applications.

In a further embodiment, amplification methods are used in conjunction with nucleic acid detection methods, including full genome sequencing methods and applications directed to detecting certain genes or certain genetic abnormalities or variations. Such nucleic acid detection methods are known in the art and can include quantitative fluorescent PCR, constant denaturant capillary electrophoresis, cycling temperature capillary electrophoresis, HPLC, as well as next generation high throughput sequencing methods utilizing either or both array based and single molecule technologies, mass spectrometry, polony sequencing, pyrosequencing, de novo sequencing technologies, shot-gun sequencing, digital PCR, as well as any other quantitative technology capable of detecting or reading DNA sequences.

In a still further embodiment, amplification methods are used in conjunction with molecular diagnostic tests. For example, amplification methods of the present invention can be used to prepare nucleic acid samples for use in the detection of genetic abnormalities using Tandem SNPs, as described for example in U.S. application Ser. No. 11/713,069, filed Feb. 28, 2007, 12/581,083, filed Oct. 16, 2009 and Ser. No. 12/581,070, filed Oct. 16, 2006, each of which is herein incorporated by reference in its entirety for all purposes and in particular for all teachings, figures, examples and data related to methods of detecting genetic abnormalities using Tandem SNPs.

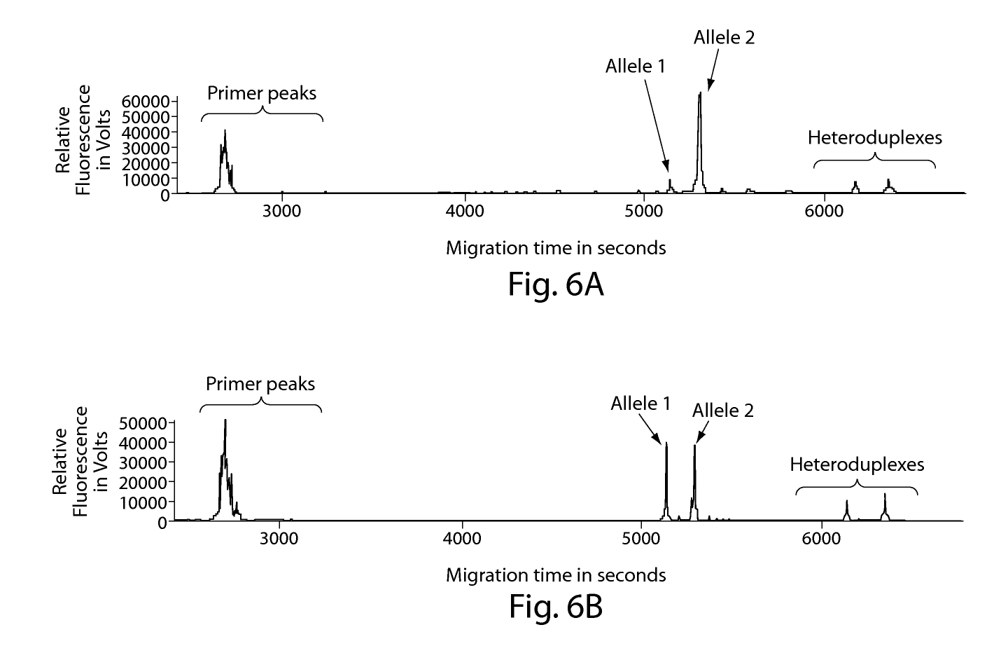

Amplification primers and methods are of particular use in whole genome amplification methods. Conventional whole genome amplification methods utilize ligation-mediated PCR methods that often introduce allelic bias. Amplification primers of the present invention, in contrast, minimize and/or completely prevent allelic bias (see FIG. 6). As such, the amplification primers can be used to amplify one or more whole genomes without allelic bias.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Analysis of Allelic Ratios After HYSA Amplification

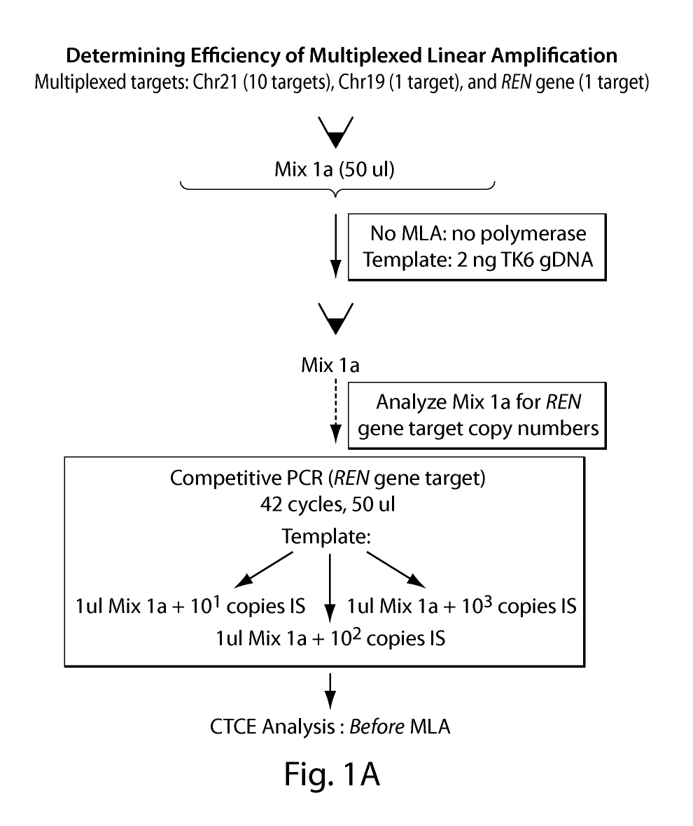

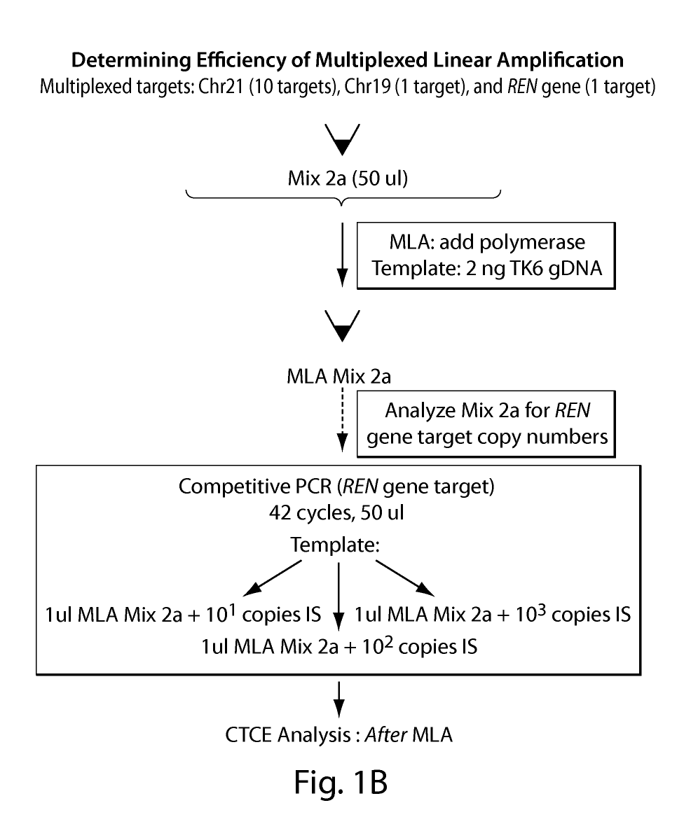

The efficiency of HYSA amplification was determined by assessing the copy number of the target sequences prior to and following HYSA amplification. FIG. 1 illustrates a HYSA amplification experimental set up. A multiplexed linear PCR was set up with 12 primer sequences targeting 12 different targets including ten tandem SNP target sequences from chromosome 21, one target from chromosome 19, and one target on exon 1 of the REN gene. 2 ng genomic DNA from TK6 cells was used as template. (a) Mix la included template, buffer, and all primers but no polymerase was added and did not undergo linear amplification and denoted as a “before MLA” mix. (b) Mix 2a was identical to Mix la except for the addition of polymerase and 45 cycles of linear amplification and is denoted as an “after MLA” mix. 1 μl of each mix was then quantified for copy numbers of the REN gene target sequence by competitive PCR using an artificial mutant sequence spiked in at three different concentrations (10¹, 10², and 10³ copies) followed by Cycling Temperature Capillary Electrophoresis (CTCE) analysis using the MegaBACETM system, a 96-capillary DNA sequencer (GE Healthcare Bio-Sciences Corp. Piscataway, N.J.). All “before MLA” and “after MLA” mixes were set up in triplicate (mix 1a, 1b, and 1c did not include polymerase and did not undergo cycling, similarly, mix 2a, 2b, and 2c did include polymerase and did undergo cycling). All competitive PCR reactions were performed in triplicate for all six mixes. Comparison of REN gene target copy numbers before and after linear amplification divided by the number of cycles led to estimation of efficiency of linear amplification (see Table 3).

TABLE 3
Data from linear amplification experiments

| | Area under | | | |
|---|---|---|---|---|
| | Wild Type | | | |
| | peak | Area under Mutant | | WT |
| | (volt-seconds) | peak (volt-seconds) | (WT):(MT) | (copies) |
| Before MLA¹ | | | | |
| Mix 1a (1 μl) + 10 | 291 | 376 | 0.77:1 | 7.7 |
| copies of IS | | | | |
| Mix 1a (1 μl) + 10 | 24,410 | 69,518 | 0.35:1 | 3.5 |
| copies of IS | | | | |
| Mix 1a (1 μl) + 10 | 18,579 | 27,309 | 0.68:1 | 6.8 |
| copies of IS | | | | |
| Mix 1b (1 μl) + 10 | 1,931 | 2,400 | 0.8:1 | 8 |
| copies of IS | | | | |
| Mix 1b (1 μl) + 10 | 835 | 591 | 1.4:1 | 14 |
| copies of IS | | | | |
| Mix 1b (1 μl) + 10 | 2,013 | 5,533 | 0.36:1 | 3.6 |
| copies of IS | | | | |
| Mix 1c (1 μl) + 10 | 7,579 | 15,656 | 0.48:1 | 4.8 |
| copies of IS | | | | |
| Mix 1c (1 μl) + 10 | 2,201 | 1,632 | 1.34:1 | 13.4 |
| copies of IS | | | | |
| Mix 1c (1 μl) + 10 | 2,874 | 1,479 | 1.94:1 | 19.4 |
| copies of IS | | | | |
| | | Average (WT:MT copies) = 9.02:10 | | |
| After MLA | | | | |
| MLA mix 2a (1 μl) + | 1,23,217.5 | 1,15,683.5 | 1.06:1 | 1,060 |
| 1,000 copies of IS | | | | |
| MLA mix 2a | 26,127.50 | 20,454.50 | 1.3:1 | 1,300 |
| (1 μl) + 1,000 copies of | | | | |
| IS | | | | |
| MLA mix 2a | 17,829 | 10,689 | 1.7:1 | 1,700 |
| (1 μl) + 1,000 copies of | | | | |
| IS | | | | |
| MLA mix 2b | 1,89,877.5 | 3,74,70.5 | 5.07:1 | 5,070 |
| (1 μl) + 1,000 copies of | | | | |
| IS | | | | |
| MLA mix 2b | 1,35,500 | 27,385 | 4.94:1 | 4,940 |
| (1 μl) + 1,000 copies of | | | | |
| IS | | | | |
| MLA mix 2b | 1,14,898 | 27,681 | 4.15:1 | 4,150 |
| (1 μl) + 1,000 copies of | | | | |
| IS | | | | |
| MLA mix 2c | 23,500 | 85,461 | 0.27:1 | 270 |
| (1 μl) + 1,000 copies of | | | | |
| IS | | | | |
| MLA mix 2c | 37,722.50 | 108202.5 | 0.34:1 | 340 |
| (1 μl) + 1,000 copies of | | | | |
| IS | | | | |
| MLA mix 2c | 1,144 | 2,640 | 0.43:1 | 430 |
| (1 μl) + 1,000 copies of | | | | |
| IS | | | | |

Average (WT:MT copies) = 2,140:1,000
Amplification = 47.35 copies per cycle
Yield = 524.9%
WT, Wild type;
MT, Mutant;
WT copies, Wild Type copies;
MLA, Multiplexed Linear Amplification.
¹Mix 1a (1ul) + 10 copies of IS, indicate that the template is 1ul from “Mix 1a” tube plus volume (equivalent to 10 copies) of internal standard (IS) sequences. Similarly the others. Before MLA, wild type copies are approximately 9. There is an amplification of 47.35 copies per cycle due MLA resulting in a yield of 524.9%.

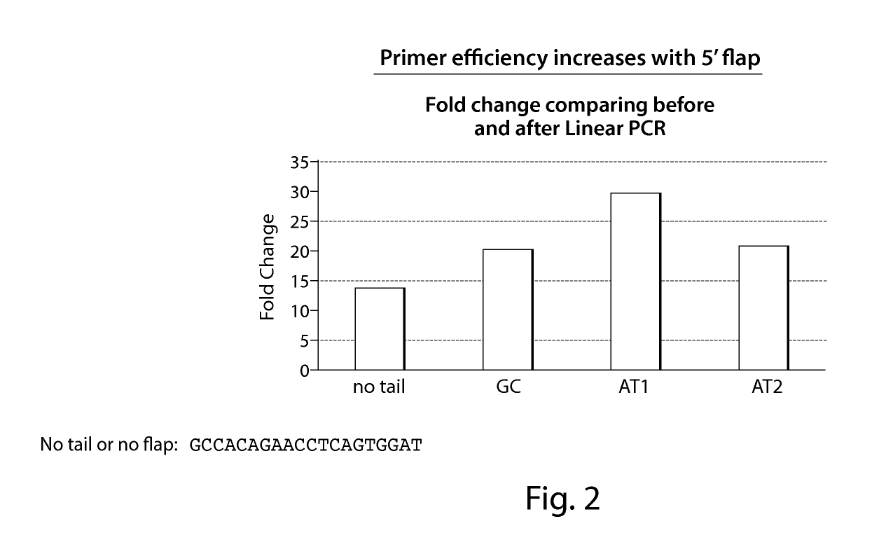

The copy number of the human renin gene was assessed using competitive PCR where a known amount of an artificial mutant PCR product containing a single basepair difference from the wild-type was added to a renin gene sequence-specific Master mix to serve as an internal standard to 1 μl of the control sample (“before HYSA”) or 1 μl of the experimental sample (“after HYSA”). The internal standard sequences were spiked in at 10 copies, 100 copies and 1000 copies per reaction to enable quantification of DNA copies before and after HYSA amplification. After amplification, CTCE analysis was performed on competitive PCR products using the MegaBACE™ DNA sequencer. Analysis from CTCE demonstrated greater than 100% yields per cycle (amplification efficiency) at the renin gene locus when multiplexed with 12 GC-rich primers, as demonstrated by the data in FIG. 2. Starting from approximately 9 copies per tube, HYSA amplification produced approximately forty-seven copies per cycle, resulting in yields per cycle of greater than 500%. As shown in the data in FIG. 2, of the primers tested, AT1 had the highest yield, followed by GC and AT2, and the no 5′-flap primer had the lowest yield.

In a separate experiment, several primers were compared to evaluate the amplification efficiency of different 5′ flap sequences. Linear amplification of the rennin gene locus was performed using these various primer sequences (AT1, GC, and AT2) and measured by quantitative real-time PCR. As shown in the data in FIG. 2, of the primers tested, AT1 had the highest yield, followed by GC and AT2, and the primer with no 5′ flap had the lowest yield.

Multiplexed linear PCR results were then compared to samples amplified by ligation-mediated PCR (LM-PCR), a commonly used technique for whole-genome amplification. As shown in FIG. 6, significant allelic differences were observed following LM-PCR in all assays, when starting with the same amounts of DNA. However, following HYSA amplification, no significant allelic difference was observed. The results from an LM-PCR experiment are shown in FIG. 6A. Starting from 6.25 ng genomic DNA, an electropherogram of a heterozygous sequence following ligation mediated PCR shows that allele 2 was clearly preferentially amplified. In contrast, with the same starting concentration of genomic DNA, an electropherogram of a heterozygous sequence following HYSA amplification shows that no observable allelic bias was present (FIG. 6B).

Without being bound to a particular mechanism, it is thought that the lack of allelic bias in HYSA amplification is that the addition of the 5′ GC-rich flap permits “unzipping” of the previously double-stranded template.

### Example 2

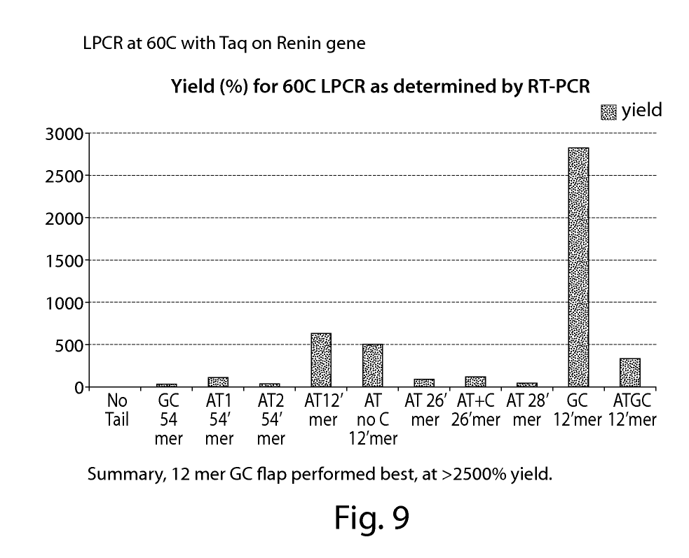

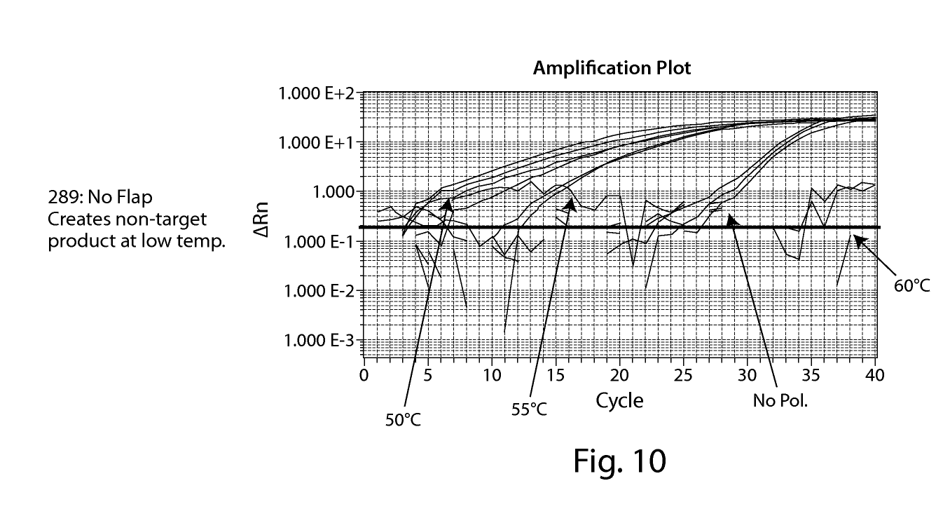

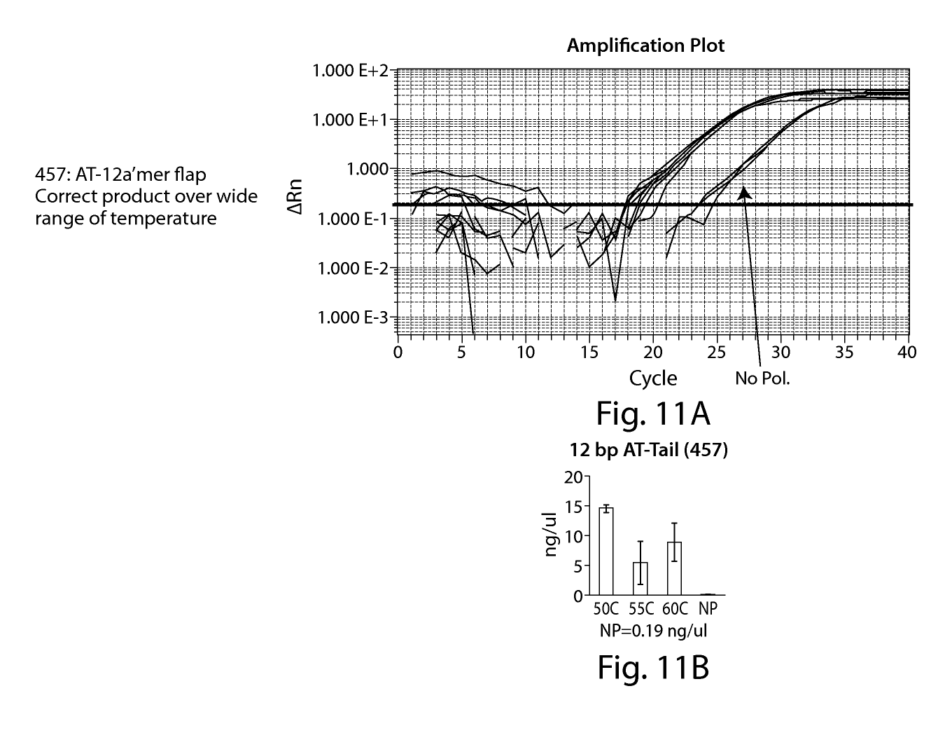

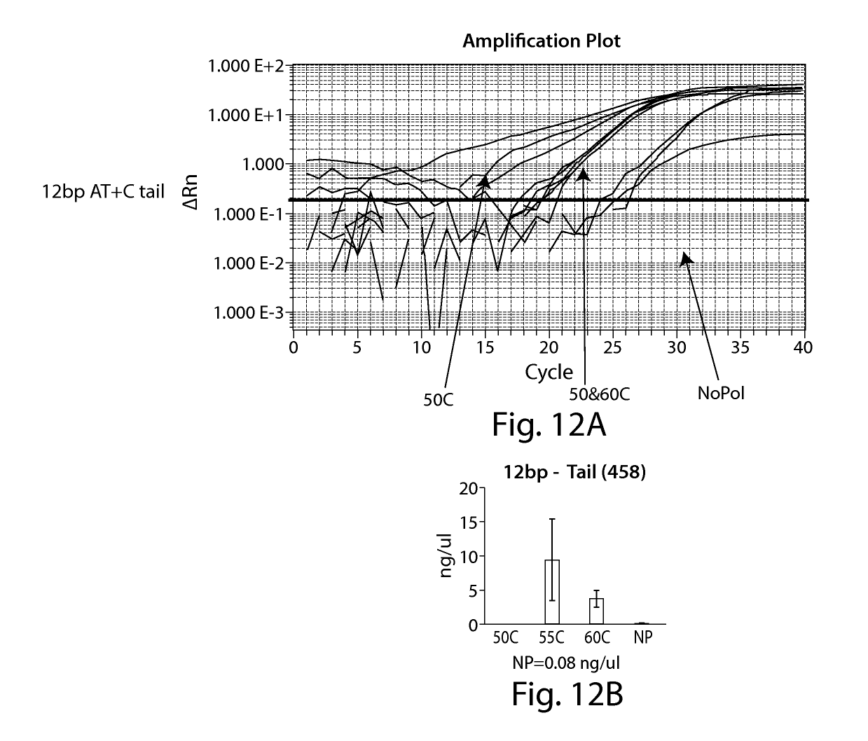

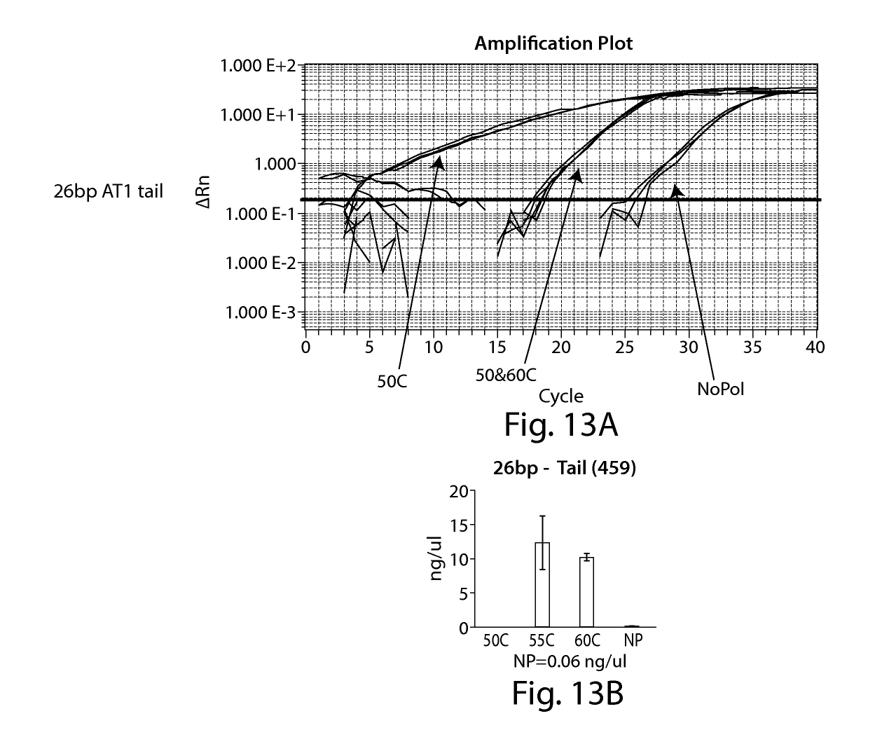

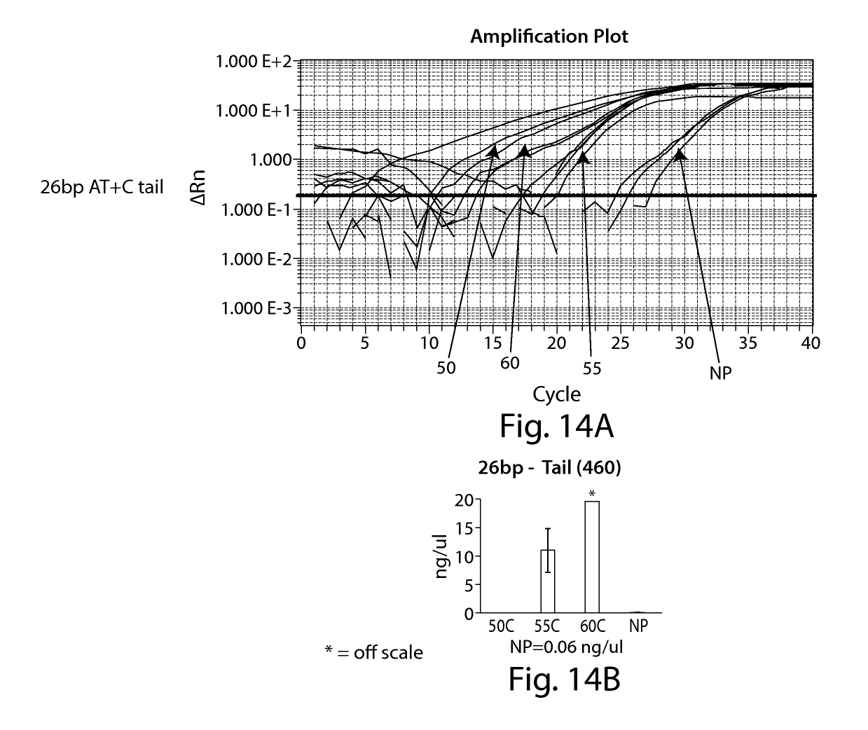

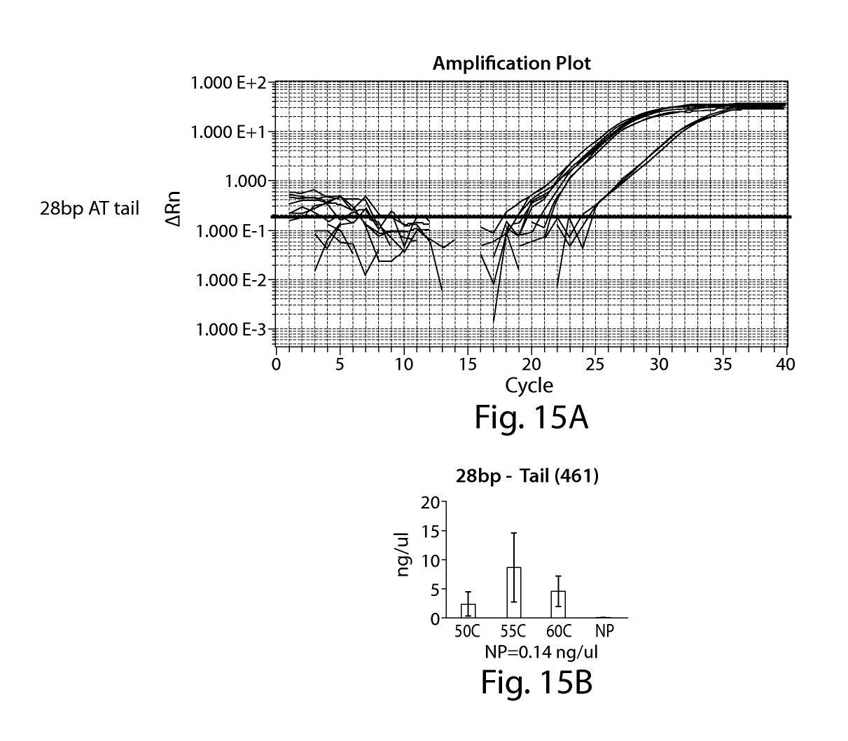

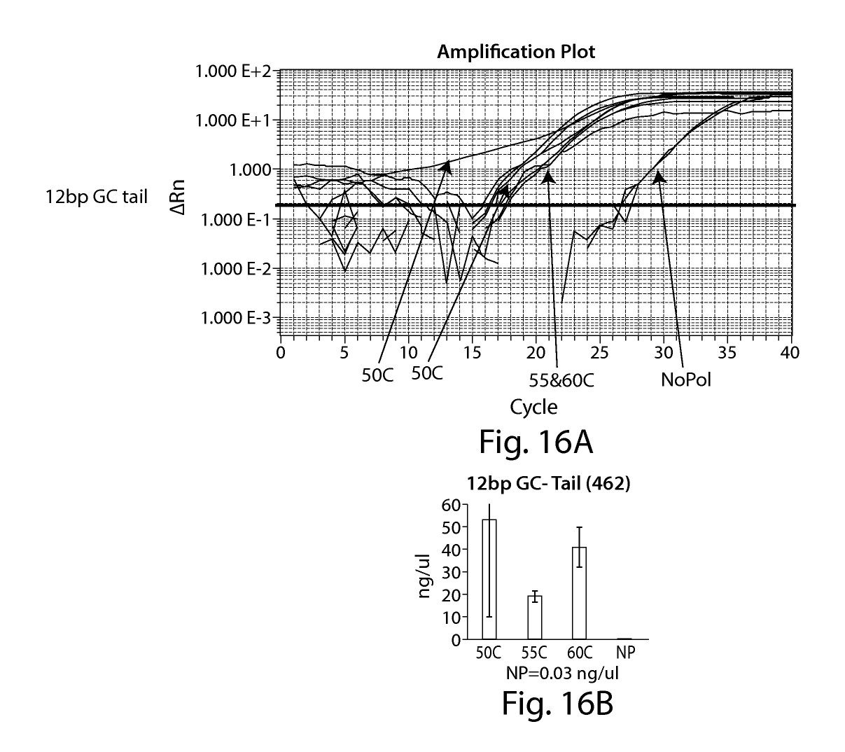

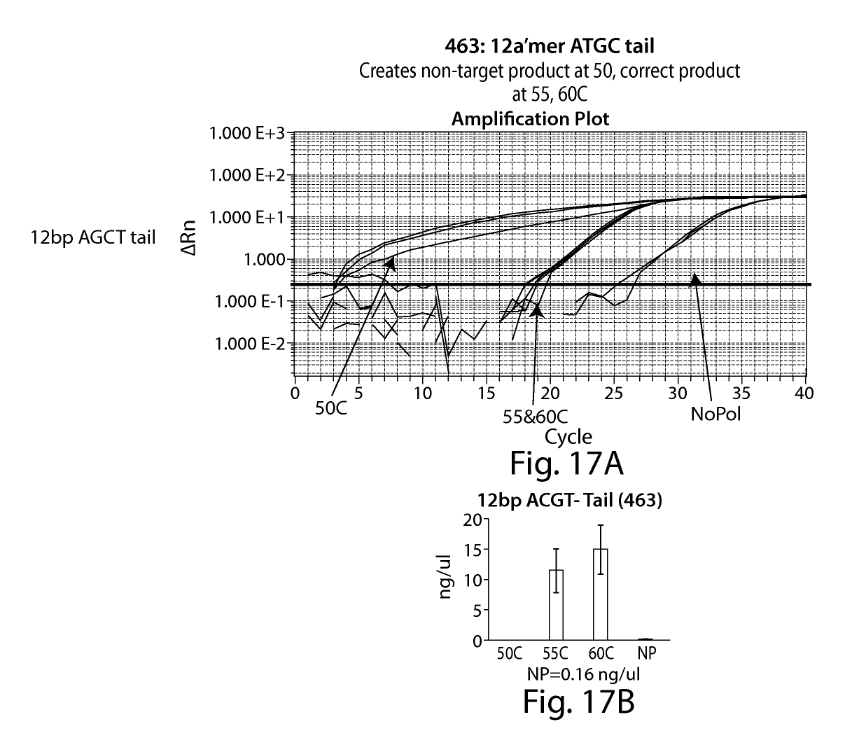

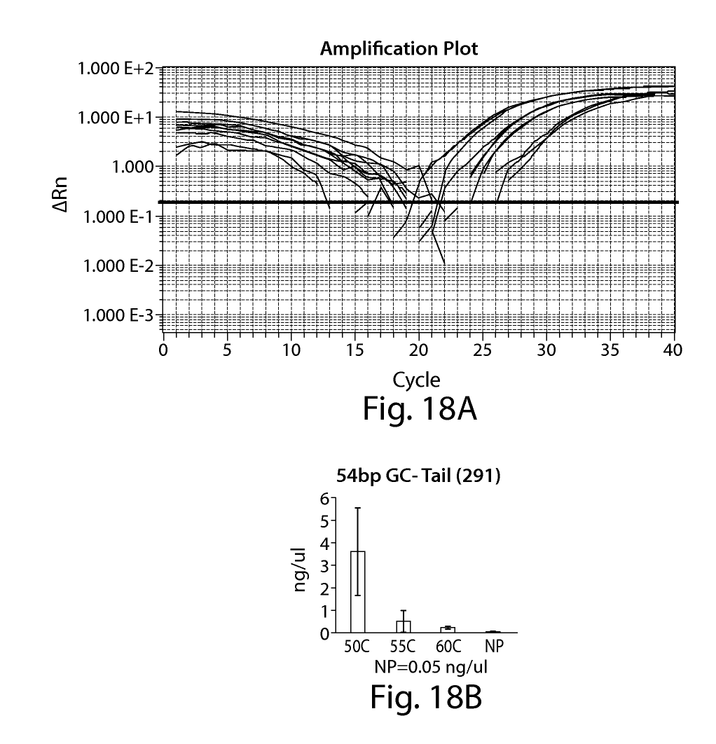

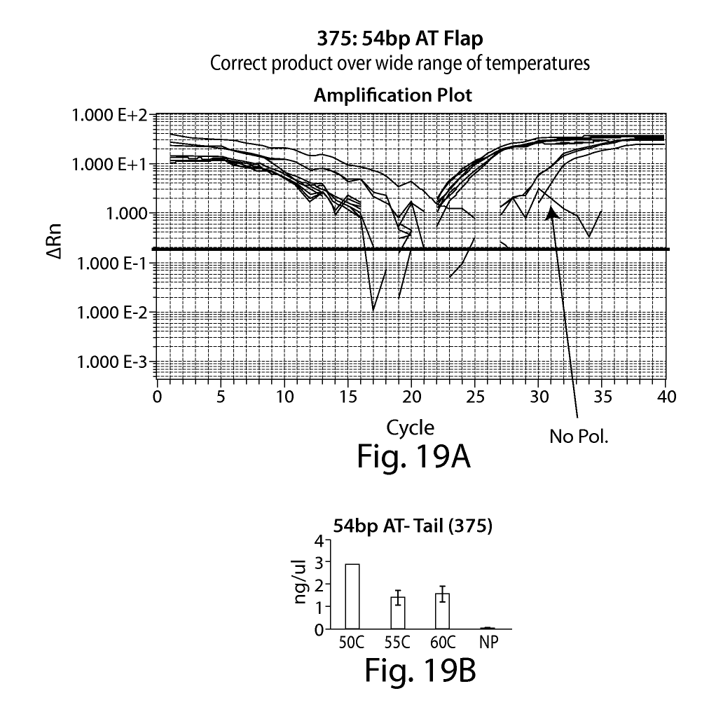

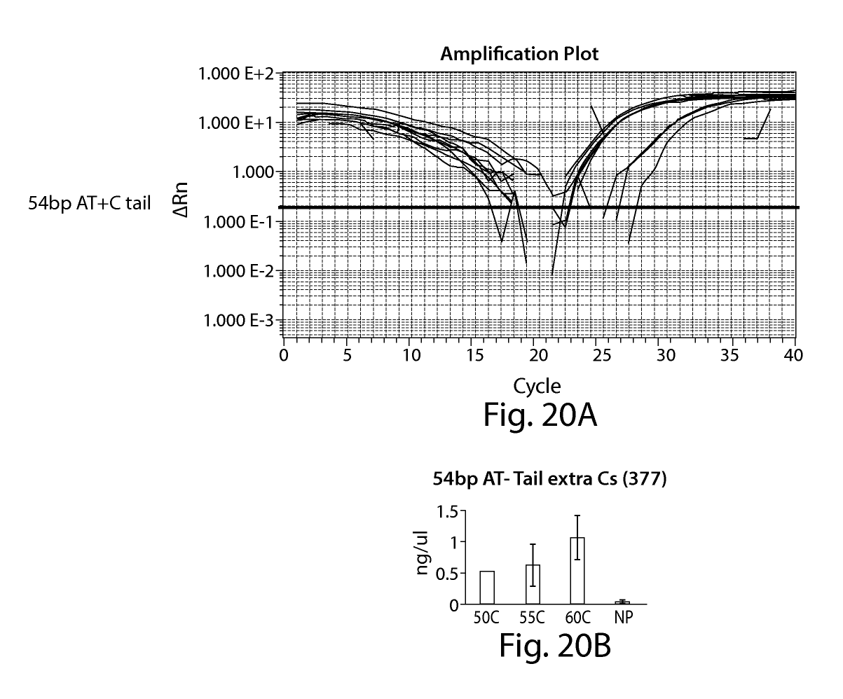

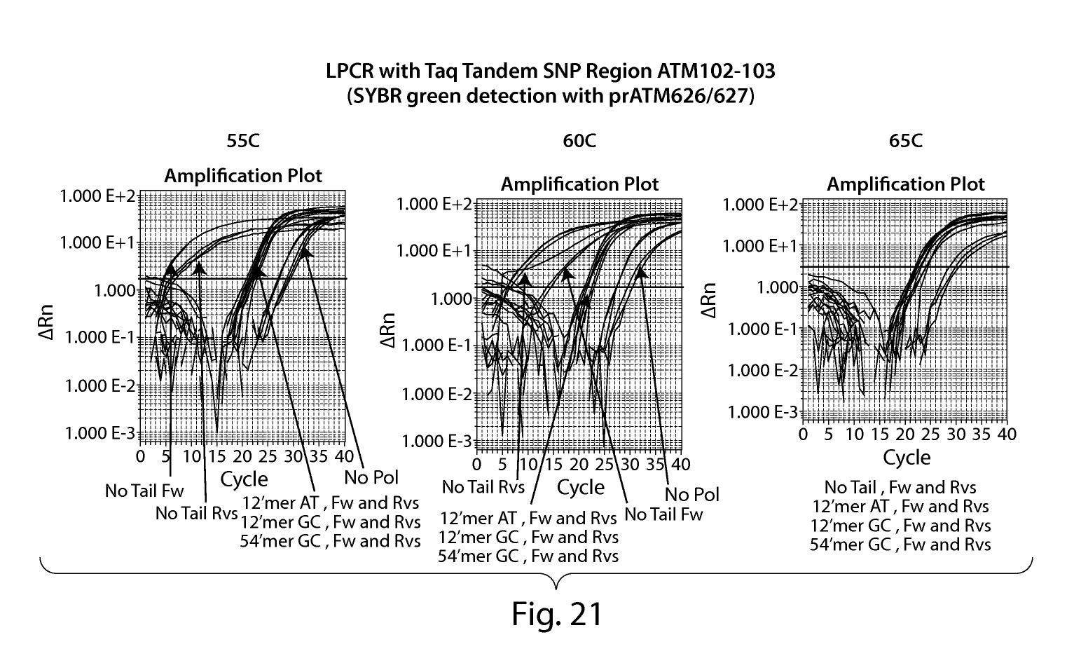

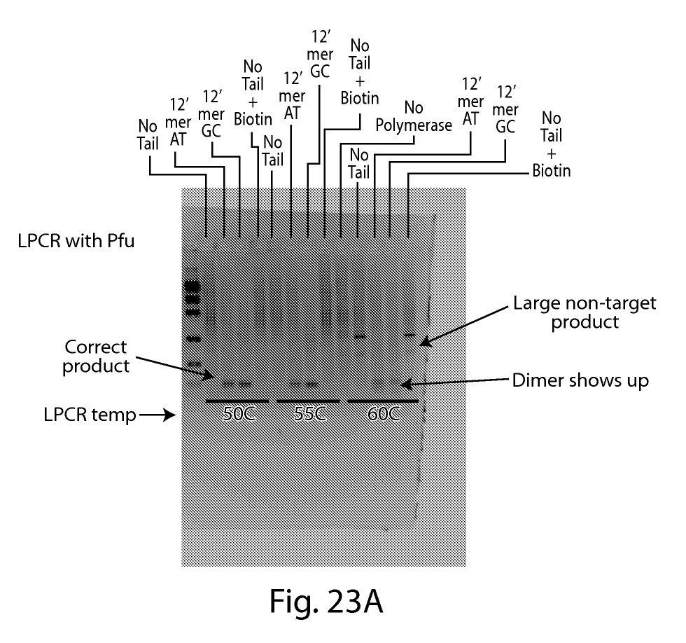

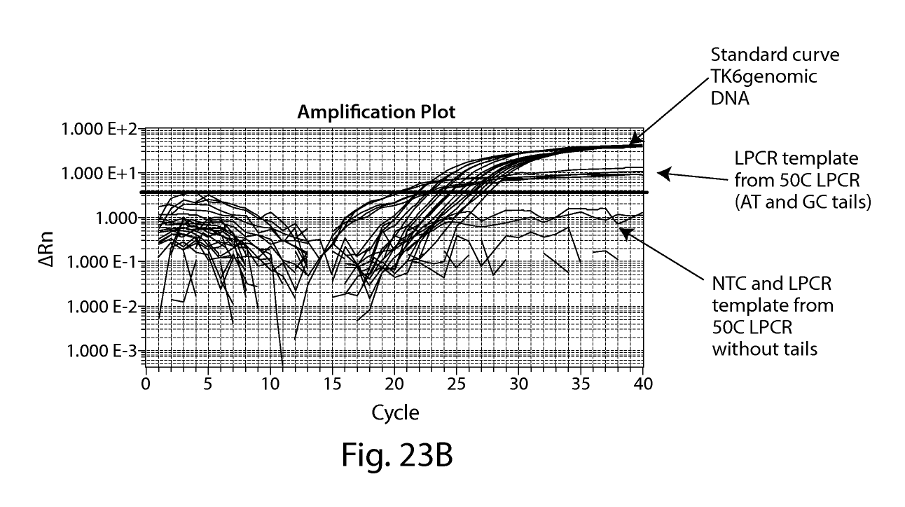

An experiment was set up to measure the yield of primers with 5′flaps of varying length and composition as shown in Table 4 following linear amplification of genomic DNA. The increase of DNA was determined by real time PCR using sequence specific primers for the same genomic region as the primer used in the linear PCR reaction. In addition the size of the amplified products was determined by agarose gel electrophoreses (2% agarose gel). The regions presented below are for the renin gene (FIGS. 9-20) and for a tandem SNP region of chromosome 21 (FIGS. 21-24).

### Materials and Methods

Starting from 10 ng of genomic DNA, 45 cycles of linear PCR reaction was performed in 50 ul total volume using 0.6 uM as a final primer concentration. Three different polymerases were tested: Taq, pfu Ultrall and Phusion. Cycling condition was set according to manufacturer recommendations. The primer sequences used were as follows (Table 4). Several annealing temperatures between 50° C. and 65° C. were tested.

TABLE 4
Primer Sequences

GC 54′mer [291]:
gccgcctgcagcccgcgccccccgtgcccccgccccgccgccggcccggg
cgcc(SEQ ID NO: 15)
AT1 54′mer [375]:
AttattCatGatttatattttttaCatttttattttattattaatttaaa
tattNNNNNNNNNNNN (SEQ ID NO: 16)
AT2 54′mer [377]:
attatttattttatttttgattatttatttatttttcattatttatttta
tttttNNNNNNNNNNNNN (SEQ ID NO: 17)
AT 12′mer [457]:
aataaatcataaNNNNNNNNNNN (SEQ ID NO: 21)
AT no C 12′mer [458]:
aataaataataaNNNNNNNNNNN (SEQ ID NO: 25)
AT 26′mer [459]:
attattcatgatttatattttttacaNNNNNNNNNNNNN
(SEQ ID NO: 18)
AT + C 26′mer [460]:
attattcatgctttatattttttacaNNNNNNNNNNNNNN
(SEQ ID NO: 19)
AT28′mer [461]:
attatgcatattttatattttttacattNNNNNNNNNNNNN
(SEQ ID NO: 20)
GC 12′mer [462]:
gccgcctgcacgNNNNNNNNNNN (SEQ ID NO: 23)
ATGC 12′mer [463]:
gtcacgtatcgaNNNNNNNNNNNNN (SEQ ID NO: 24)

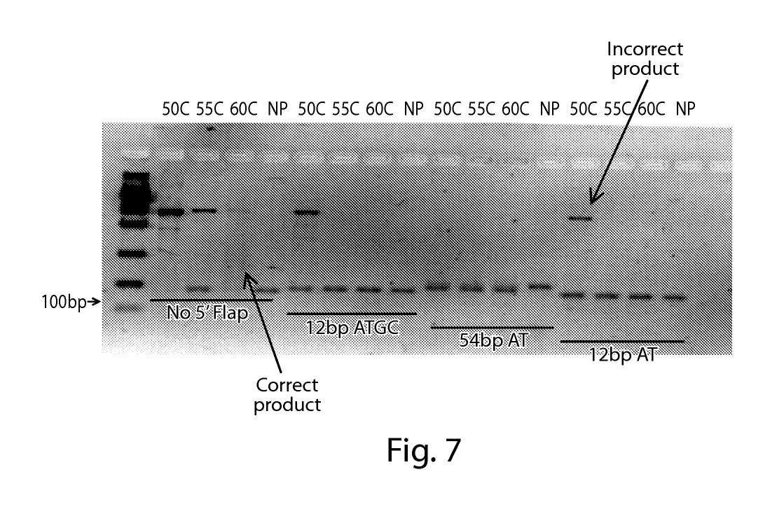

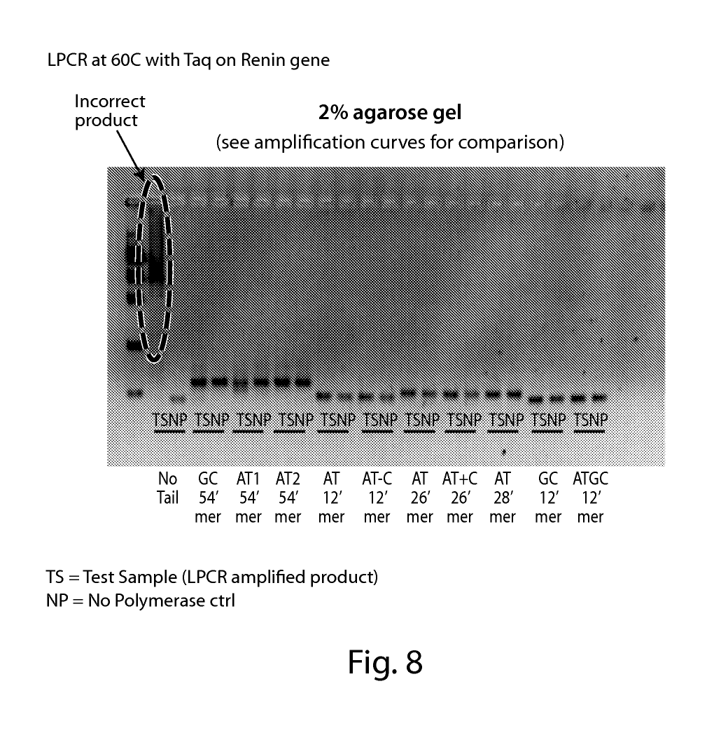

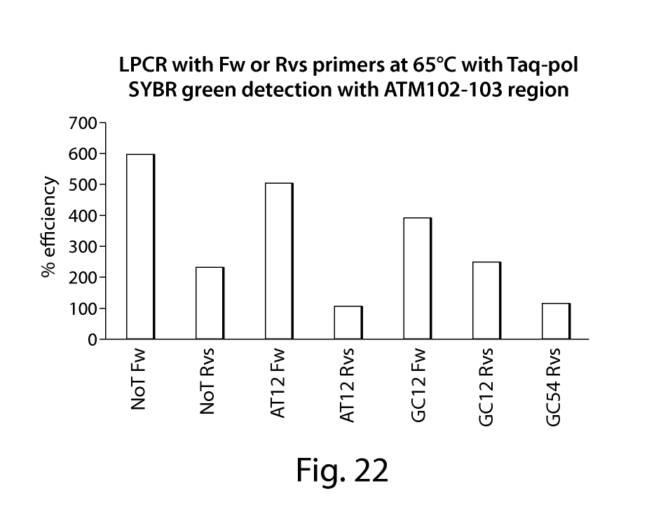

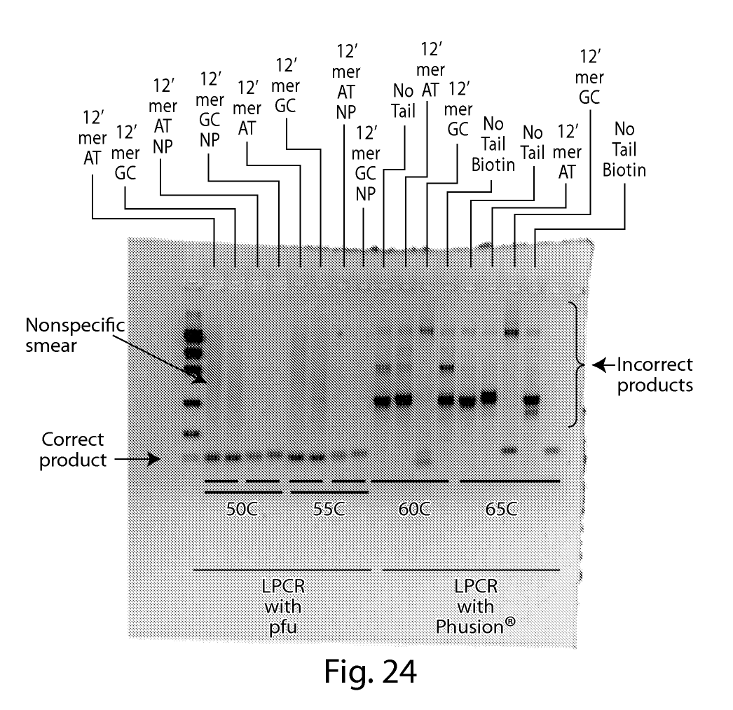

Subsequently, a sequence specific PCR was performed using the 5′ flap primer or with no tail primer and visualized by real-time PCR (SYBR green). Yield of a linear PCR reaction was determined by comparing the fold-increase of a linear PCR reaction with polymerase to the linear PCR reaction without any polymerase (no polymerase) using real-time PCR where the linear PCR reaction was used as a template, and dividing by the number of cycles used in a linear PCR reaction. The products were also run on a 2% agarose gel to determine if product size was correct. It was found that GC 12′mer provided a yield of greater than 2500% (FIG. 9) where the resulting PCR product demonstrated an expected size as determined by agarose gel analysis. With respect to the other primers, the ones without tails only worked at temperatures above 60° C. or higher and often not at all. At lower temperatures, these primers always yielded non-target, undesirable high molecular weight products (FIGS. 7, 8, 10 and 21). Tails can “rescue” the linear PCR reaction to be more specific but the yield varies depending on length and composition as summarized in FIG. 9. As stated above, the GC12′mer demonstrated high yield at 60° C. In addition, it is less sensitive to sequence specificity (FIG. 22, performed with a high annealing temperature of 65° C., demonstrates linear PCR reactions with no flap or tail, the 12-mer AT flap, and the 12-mer GC flap, and the 54-mer GC flap, using both forward or reverse primers, where significantly higher yield for the forward primer is seen for the no tail compared to the no tail for the reverse primer). Moreover the GC 12′mer was the only primer that yielded a correct product when Phusion enzyme was used (FIG. 24). In summary, 5′flaps can make the linear PCR more specific over a wide range of temperatures as determined by the formation of the correct molecular weight PCR product. In addition, the yield is increased especially if the flap has a low tendency to form hairpin loops.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for amplifying multiple target polynucleotides, comprising:
(a) adding two or more high yield, instability primers to a reaction mixture that comprises two or more target polynucleotides; and
(b) incubating the reaction mixture under conditions that promote replication of the target polynucleotides, thereby amplifying the target polynucleotides, wherein at least one of the high yield, instability primers do not comprise a GC-clamp with the sequence set forth as SEQ ID NO: 15.

2. The method of claim 1, wherein at least one of the high yield, instability primers comprise an oligonucleotide flap.

3. The method of claim 2, wherein the oligonucleotide flap is a 5′ flap.

4. The method of claim 2, wherein the oligonucleotide flap is an AT-rich flap.

5. The method of claim 2, wherein the oligonucleotide flap is a GC-rich flap.

6. The method of claim 5, wherein the oligonucleotide flap is a mismatched sequence relative to a target sequence.

7. The method of claim 2, wherein the at least one of the high yield, instability primers exhibits minimal or no self-annealing.

8. The method claim 2, wherein the oligonucleotide flap consists of fewer than 54 nucleotides.

9. The method of claim 8, wherein the oligonucleotide flap consists of fewer than 30 nucleotides.

10. The method of claim 9, wherein the oligonucleotide flap consists of fewer than 25 nucleotides.

11. The method of claim 10, wherein the oligonucleotide flap consists of fewer than 20 nucleotides.

12. The method of claim 11, wherein the oligonucleotide flap consists of fewer than 15 nucleotides.

13-29. (canceled)

30. A method for amplifying a minority sequence, comprising:
(a) adding a high yield, instability primer to a reaction mixture that comprises a target polynucleotide comprising the minority sequence; and
(b) incubating the reaction mixture under conditions that promote replication of the target polynucleotide, thereby amplifying the minority sequence, wherein the high yield, instability primer does not comprise a GC-clamp with the sequence set forth as SEQ ID NO: 15.

31-67. (canceled)

68. A method for generating an unstable target extension product, comprising:
(a) adding a high yield, instability primer to a reaction mixture that comprises a target polynucleotide template; and
(b) incubating the reaction mixture under conditions that promote replication and amplification of the target polynucleotide template, thereby generating the unstable target extension product,
wherein the high yield, instability primer is i) a non-self annealing primer comprising an oligonucleotide flap, ii) has an annealing temperature that is at or above its calculated melting temperature when Taq polymerase is added to the reaction mixture, iii) comprises one or more mismatches within its 5′ region, or iv) can anneal and amplify a target polynucleotide at more than one temperature.

69-85. (canceled)

86. A method for generating unstable target extension products, comprising:
(a) adding a high yield, instability primer to a reaction mixture that comprises a target polynucleotide template; and
(b) incubating the reaction mixture under conditions that promote replication and amplification of the target polynucleotide template, thereby generating the unstable target extension products, wherein the high yield, instability primer does not comprise a flap, and wherein the conditions include an annealing temperature that is greater than the calculated melting temperature of the primer or that is less than the calculated melting temperature of the primer (without the flap).

87-90. (canceled)

91. A method of testing a primer, comprising:
(a) adding to a terminus of the primer an oligonucleotide flap;
(b) testing the primer to determine if it anneals and amplifies a target polynucleotide at at least two different temperatures or testing the primer to determine the yield at which it anneals and amplifies at least one target polynucleotide template; and
(c) testing the primer to determine if a non-target polynucleotide extension product is produced.

92-93. (canceled)

94. A method of testing a primer, comprising:
(a) creating a primer that has an annealing temperature that is at or above its calculated melting temperature or melting temperature but that does not comprise an oligonucleotide flap;
(b) testing the primer to determine if it anneals and amplifies a target polynucleotide in the presence of Taq polymerase or at at least two different temperatures in the presence of Taq polymerase; and
(c) determining if a non-target polynucleotide extension product is produced or is produced at each of the temperatures.

95-97. (canceled)

98. A method of testing a primer, comprising:
(a) creating a primer that comprises one or more mismatches in its 5′ region;
(b) testing the primer to determine the yield at which it anneals and amplifies at least one target polynucleotide or testing the primer to determine if it anneals and amplifies a target polynucleotide at at least two different temperatures; and
(c) determining if a non-target polynucleotide extension product is produced or determining if a non-target polynucleotide extension product is produced at each temperature.

99. (canceled)

100. A high yield, instability primer, comprising 1) an oligonucleotide flap at one terminus, wherein when added to a reaction mixture comprising a target polynucleotide, under conditions that permit replication and amplification of the target polynucleotide, the primer exhibits no self-annealing; 2) an oligonucleotide flap at one terminus of the primer, wherein when added to a reaction mixture comprising a target polynucleotide, under conditions that permit replication and amplification of the target polynucleotide: (a) a target extension product is produced at a yield of greater than 100%; and (b) no non-target extension product is produced; 3) one or more mismatches within its 5′ region, wherein when added to a reaction mixture comprising a target polynucleotide and a polymerase, under conditions that permit replication and amplification of the target polynucleotide, target extension product is produced; or 4) a sequence selected from the group of sequences set forth as SEQ ID NOs: 1-14.

101-110. (canceled)

111. A primer that has an annealing temperature that is at or above its calculated melting temperature, wherein when added to a reaction mixture comprising a target polynucleotide and Taq polymerase, under conditions that permit replication and amplification of the target polynucleotide, target extension product is produced.

112-120. (canceled)


SEQUENCE LISTING (32 sequences)

SEQ ID NO: 1 - DNA, 74 bp, Artificial Sequence
  oligonucleotide
gccgcctgcagcccgcgccccccgtgcccccgccccgccgccggcccgggcgcccagtgtttggaaattgtctg

SEQ ID NO: 2 - DNA, 72 bp, Artificial Sequence
  oligonucleotide
gccgcctgcagcccgcgccccccgtgcccccgccccgccgccggcccgggcgcctcttccaccacaccaatc

SEQ ID NO: 3 - DNA, 74 bp, Artificial Sequence
  oligonucleotide
gccgcctgcagcccgcgccccccgtgcccccgccccgccgccggcccgggcgccaaaagatgagacaggcaggt

SEQ ID NO: 4 - DNA, 72 bp, Artificial Sequence
  oligonucleotide
gccgcctgcagcccgcgccccccgtgcccccgccccgccgccggcccgggcgccgtgggagcactgcaggta

SEQ ID NO: 5 - DNA, 75 bp, Artificial Sequence
  oligonucleotide
gccgcctgcagcccgcgccccccgtgcccccgccccgccgccggcccgggcgccaattattattttgcaggcaat

SEQ ID NO: 6 - DNA, 76 bp, Artificial Sequence
  oligonucleotide
gccgcctgcagcccgcgccccccgtgcccccgccccgccgccggcccgggcgccttcaaattgtatataagagagt

SEQ ID NO: 7 - DNA, 76 bp, Artificial Sequence
  oligonucleotide
gccgcctgcagcccgcgccccccgtgcccccgccccgccgccggcccgggcgccattgttagtgcctcttctgctt

SEQ ID NO: 8 - DNA, 74 bp, Artificial Sequence
  oligonucleotide
gccgcctgcagcccgcgccccccgtgcccccgccccgccgccggcccgggcgcctcagacttgaagtccaggat

SEQ ID NO: 9 - DNA, 81 bp, Artificial Sequence
  oligonucleotide
gccgcctgcagcccgcgccccccgtgcccccgccccgccgccggcccgggcgccaaggatagagatatacagatgaatgga

SEQ ID NO: 10 - DNA, 74 bp, Artificial Sequence
  oligonucleotide
gccgcctgcagcccgcgccccccgtgcccccgccccgccgccggcccgggcgccatagcctgtgagaatgccta

SEQ ID NO: 11 - DNA, 20 bp, Artificial Sequence
  oligonucleotide
gccacagaacctcagtggat

SEQ ID NO: 12 - DNA, 74 bp, Artificial Sequence
  oligonucleotide
gccgcctgcagcccgcgccccccgtgcccccgccccgccgccggcccgggcgccgccacagaacctcagtggat

SEQ ID NO: 13 - DNA, 74 bp, Artificial Sequence
  oligonucleotide
attattcatgatttatattttttacatttttattttattattaatttaaatattgccacagaacctcagtggat

SEQ ID NO: 14 - DNA, 75 bp, Artificial Sequence
  oligonucleotide
attatttattttatttttgattatttatttatttttcattatttattttatttttgccacagaacctcagtggat

SEQ ID NO: 15 - DNA, 54 bp, Artificial Sequence
  oligonucleotide
gccgcctgcagcccgcgccccccgtgcccccgccccgccgccggcccgggcgcc

SEQ ID NO: 16 - DNA, 54 bp, Artificial Sequence
  oligonucleotide
attattcatgatttatattttttacatttttattttattattaatttaaatatt

SEQ ID NO: 17 - DNA, 55 bp, Artificial Sequence
  oligonucleotide
attatttattttatttttgattatttatttatttttcattatttattttattttt

SEQ ID NO: 18 - DNA, 26 bp, Artificial Sequence
  oligonucleotide
attattcatgatttatattttttaca

SEQ ID NO: 19 - DNA, 26 bp, Artificial Sequence
  oligonucleotide
attattcatgctttatattttttaca

SEQ ID NO: 20 - DNA, 28 bp, Artificial Sequence
  oligonucleotide
attatgcatattttatattttttacatt

SEQ ID NO: 21 - DNA, 12 bp, Artificial Sequence
  oligonucleotide
aataaatcataa

SEQ ID NO: 22 - DNA, 9 bp, Artificial Sequence
  oligonucleotide
aaatcataa

SEQ ID NO: 23 - DNA, 12 bp, Artificial Sequence
  oligonucleotide
gccgcctgcacg

SEQ ID NO: 24 - DNA, 12 bp, Artificial Sequence
  oligonucleotide
gtcacgtatcga

SEQ ID NO: 25 - DNA, 12 bp, Artificial Sequence
  oligonucleotide
aataaataataa

SEQ ID NO: 26 - DNA, 15 bp, Artificial Sequence
  oligonucleotide
tataataaatcataa

SEQ ID NO: 27 - DNA, 18 bp, Artificial Sequence
  oligonucleotide
caggaaacagctatgacc

SEQ ID NO: 28 - DNA, 18 bp, Artificial Sequence
  oligonucleotide
tgtaaaacgacggccagt

SEQ ID NO: 29 - DNA, 20 bp, Artificial Sequence
  oligonucleotide
attaaccctcactaaaggga

SEQ ID NO: 30 - DNA, 20 bp, Artificial Sequence
  oligonucleotide
taatacgactcactataggg

SEQ ID NO: 31 - DNA, 3 bp, Artificial Sequence
  oligonucleotide
taa

SEQ ID NO: 32 - DNA, 6 bp, Artificial Sequence
  oligonucleotide
tcataa